(19) United States
(12) Patent Application Publication
(10) Pub. No.: US 2024/0148679 A1
(43) Pub. Date: May 9, 2024

(54) METHODS AND COMPOSITIONS RELATED TO TREATMENT AND PREVENTION OF CANCER BY INHIBITION OF DGAT1 AND DGAT2

(71) Applicant: Ohio State Innovation Foundation, Columbus, OH (US)

(72) Inventor: Deliang GUO, Dublin, OH (US)

(21) Appl. No.: 17/769,512

(22) PCT Filed: Oct. 15, 2020
(86) PCT No.: PCT/US2020/055858

§ 371 Date: Apr. 15, 2022

Related U.S. Application Data
(60) Provisional application No. 62/915,760, filed on Oct. 16, 2019.

(51) Int. Cl. A61K 31/196; A61K 31/4545; A61P 35/00

(52) U.S. Cl. CPC A61K 31/196; A61K 31/4545; A61P 35/00

(57) ABSTRACT

Disclosed herein are compounds, compositions, and methods for inhibiting DGAT1 and DGAT2. Also disclosed are kits comprising DGAT1 and DGAT2 inhibitors. The compounds, compositions, and methods can be used to treat a subject with cancer, such as liver cancer.

### CROSS-REFERENCE TO RELATED APPLICATIONS

This application claims benefit of U.S. Provisional Application No. 62/915,760, filed Oct. 16, 2019, which is hereby incorporated by reference in its entirety.

### STATEMENT REGARDING FEDERALLY SPONSORED RESEARCH OR DEVELOPMENT

This invention was made with government support under Grant Nos. R01NS079701 and R01NS104332 awarded by the National Institute of Neurological Disorders and Stroke, and R01CA227874 awarded by the National Cancer Institute. The government has certain rights in the invention.

### BACKGROUND

Fatty acids are essential lipids in cells. They constitute the major structural components of membrane lipids, i.e., glycerophospholipids and sphingolipids, and also serve as an important energy resource through mitochondria-mediated β-oxidation and tricarboxylic acid (TCA) cycle catabolism (Carracedo 2013; Currie 2013; Qu 2016). However, excess fatty acids or accumulation of intermediates of fatty acid metabolism can cause cytotoxicity, leading to cell damage (Ertunc 2016; Listenberger 2003). Thus, controlling free fatty acid levels and sustaining their homeostasis by channeling them into mitochondria for oxidation and to structural lipids are critical to maintain cellular function.

In cancer cells, many studies have demonstrated that lipid metabolism is significantly altered, especially fatty acid synthesis, which is greatly elevated in various types of cancers (Cheng 2018; Guo 2014; Menendez 2007). However, how lipid homeostasis is maintained in tumor cells to prevent excessive fatty acids from causing toxicity is unknown. It was recently observed that lipid droplets (LDs), the specific lipid-storage organelles, are prevalent in glioblastoma (GBM) (Geng 2016; Geng 2017), the most lethal primary brain tumor (Wen 2016). LDs are also observed in prostate, colon and renal cancers (Yue 2014; Qui 2015; Accioly 2008). Nevertheless, the role of LDs in cancer cells has remained unclear. It is known that LDs are commonly formed in adipose tissues to store excess fatty acids (Walther 2012). They are also formed in hepatocytes and macrophages in patients with fatty liver or atherosclerosis (Krahmer 2013). LDs store large quantities of triglycerides (TG) and cholesteryl esters, which are surrounded by a phospholipid monolayer that integrates various proteins (Walther 2012). When fatty acids are in excess in the body, they are absorbed by adipocytes in adipose tissues and converted to TG. The final and committed step for TG synthesis is regulated by two diacylglycerol-acyltransferases, DGAT1 and DGAT2 (Coleman 2004; Coleman 2011). These enzymes, which are transmembrane proteins residing in the endoplasmic reticulum (ER), catalyze the esterification of fatty acid-CoA, also named acyl-CoA, with diacylglycerol (DAG) to form TG, which are then assembled and bud from the ER into the cytosol to form LDs (Yen 2008).

DGAT1 and DGAT2 have different distributions in human tissues. DGAT1 is highly expressed in the intestine to promote fatty acid absorption, and is also expressed in the testis, adipose tissues and liver (Cases 1998). Expression of DGAT2 is high in the liver and adipose tissues, but low in the intestine (Cases 2011). However, what is needed in the art is an understanding of the function of DGAT1 and DGAT2 in human cancers.

### SUMMARY

Disclosed herein is a method of treating cancer in a subject in need thereof, the method comprising administering to the subject an inhibitor of DGAT1, and an inhibitor of DGAT2, or a combination of both. The subject can have liver cancer, and can be diagnosed with liver cancer prior to treatment. The subject can be given a combination of a DGAT1 and a DGAT2 inhibitor. The DGAT1 inhibitor can be Pradigastat and/or A922500, T863, AZD-7687, or AZD 3988. The DGAT2 inhibitor can be PF06424439 and/or JNJ DGAT2-A. The DGAT1 and DGAT2 inhibitors can be given simultaneously or sequentially, such as within 24 or 48 hours. In addition to DGAT1 and/or DGAT2 inhibition, the subject can also be treated with a further liver cancer treatment method such as surgery, ablation, embolization, radiation, targeted therapy, chemotherapy, and immunotherapy. The subject can be monitored during treatment and the treatment is adjusted accordingly.

Also disclosed herein is a kit comprising a DGAT1 inhibitor and a DGAT2 inhibitor. The kit can comprise a means for delivering the DGAT1 and DGAT2 inhibitors to a subject in need thereof. The DGAT1 and DGAT2 inhibitors can be premixed together. The DGAT1 and DGAT2 inhibitors can be provided in containers designed to be premixed before administration to a subject.

Further disclosed is a method of treating a subject in need of receiving either DGAT1 inhibition, DGAT2 inhibition, or both, the method comprising: a) selecting a subject diagnosed with cancer; b) measuring levels of DGAT1 and DGAT2 in the subject; c) determining whether the subject is in need of DGAT1 inhibition, DGAT2 inhibition, or both; d) selecting those subjects in need of DGAT1 inhibition, DGAT2 inhibition, or both; and e) treating those subjects in need of DGAT1 inhibition, DGAT2 inhibition, or both accordingly.

Disclosed is a method of treating a subject with cancer, the method comprising administering to the subject a DGAT1 inhibitor, such as Pradigastat and/or A922500, T863, AZD-7687, or AZD 3988.

Further disclosed is a method of treating a subject with cancer, the method comprising administering to the subject a DGAT2 inhibitor such as JNJ DGAT2-A.

### BRIEF DESCRIPTION OF THE FIGURES

FIG. 1A-I shows GBM tumors contain large amounts of TG and express high levels of DGAT1 that are associated with poor patient survival. (A) Thin layer chromatography (TLC) analysis of lipids extracted from normal brains vs. GBM tissues from patients. Relative TG levels were quantified by ImageJ by measuring the density of the iodine staining (right panel). Statistical significance was determined by Student's t-test (mean±SEM, n=7). (B) Western blot analysis of DGAT1 expression in human normal brain vs. GBM tumors. Protein disulfide-isomerase family A, member 1 (PDIA1) is an ER-resident protein and was used as an internal control. (C) Immunohistochemistry (IHC) analysis of DGAT1 in human normal brain vs. GBM tumor samples (upper panels). Lipid droplets (LDs) were detected by immunofluorescence (IF) staining of the LD membrane protein marker, TIP47 (Red) (lower panels). Nucleus was stained with DAPI (blue). Scale bar, 50 m for IHC, 10 m for IF images. (D) RT-qPCR analysis of DGAT1 vs. DGAT2 mRNA levels in human GBM tumor samples (n=10). The expression was normalized to the average DGAT1 expression. Statistical significance was determined by a Student's t-test. (E) Boxplot analysis of DGAT1 vs. DGAT2 gene expression in GBM (n=153), ovarian (n=303), prostate (n=497), breast (n=1009) and liver (n=371) cancer patient samples from the TCGA RNA-seq databases. Statistical significance was determined by Student's t-test. RPKM, reads per kilobase million. (F, G) RIC analysis of DGAT1 expression in a tissue microarray (TMA) containing various human brain tumor samples (n=62) (upper panels). LDs were detected by IF via TIP47 staining (red) (lower panels). Nucleus was stained with DAPI (blue). Scale bar, 20 m for IHC, 10 m for IF in panel (F) The expression levels of DGAT1 in different tumor tissues were quantified by H-score as described in the Methods and shown in (G). Statistical significance between tumor grades was determined by one-way ANOVA. Statistical significance among tumor grades was determined by ANOVA followed by pairwise comparisons. *P<0.01. PA, pilocytic astrocytoma, grade I; A2, astrocytoma grade II; AA, anaplastic astrocytoma, grade III. (H) Kaplan-Meier plot of GBM patient overall survival stratified on the basis of DGAT1 protein levels in TMA analyzed in panels F and G (high vs. low). The mean H-score of DGAT1 expression is 180. Above the mean were grouped as high group, and less than the mean as low group. (I) Kaplan-Meier plot of GBM patient overall survival based on DGAT1 mRNA expression levels in GBM TCGA database (RNA-seq). The optimal cut-off 9.503 was applied to stratify the high vs. low groups. Statistical significance was analyzed by log-rank test for both (H) and (I).

FIG. 2A-G shows inhibition of DGAT1, but not DGAT2, significantly suppresses TG/LD formation and induces GBM cell death. (A) RT-qPCR analysis of DGAT1 vs. DGAT2 mRNA expression in GBM cells (U251 and GBM30) and in liver cancer cell line (HepG2). The results are shown as mean±SD (n=3). Statistical significance was determined by a paired Student's t-test. *P<0.01; n.s., not significant. (B) Representative fluorescence imaging of LDs stained with BODIPY 493/503 (green) (upper panels) and TLC analysis of TG levels (lower panels) in GBM and liver cancer cells after treatments with the DGAT1 inhibitor A-922500 (20 μg/ml) or DGAT2 inhibitor PF-06424439 (20 g/ml) alone or in combination for 24 hr. Nuclei were stained with Hoechst 33342 (blue). Scale bar, 10 m. LDs were quantified by the ImageJ software in more than 30 cells in each treatment group (mean±SEM). TG levels were quantified by the ImageJ software and normalized to the untreated control group (mean±SD, n=3). Statistical significance was determined by one-way ANOVA. * P<0.001; #P<0.05. (C) Percentage of dead GBM or liver cancer cells after treatment with DGAT1 or DGAT2 inhibitors alone or in combination for 3 days as in panel B. Live and dead cells were counted after trypan blue staining (mean SD, n=3). Statistical significance was determined by one-way ANOVA. * P<0.0001. (D) Western blot analysis of DGAT1 expression in membrane extracts from GBM and liver cancer cells after infection with shRNA-expressing lentivirus against DGAT1 for 48 hr. PDIA1, an ER protein marker, was used as an internal control. (E) RT-qPCR analysis of DGAT2 mRNA expression in GBM and liver cancer cells after infection with shRNA-expressing lentivirus against DGAT2 for 48 hr. The expression level was normalized to control cells and shown as mean±SD (n=3). Statistical significance was determined by one-way ANOVA. * P<0.01. (F) Representative fluorescence images of LDs stained with BODIPY 493/503 (upper panels) and TLC analysis of TG levels (lower panels) in GBM and liver cancer cells after lentivirus-mediated shRNA knockdown of DGAT1 or DGAT2 alone or in combination for 48 hr. Scale bar, 10 m. Quantification and significance analysis of LDs and TG levels in different groups were the same as in panel B. * P<0.001; #P<0.05. (G) Percentage of dead GBM or liver cancer cells after lentivirus-mediated shRNA knockdown of DGAT1 or DGAT2 alone or in combination for 4 days. Cell death percentage and significance were determined as in panel c (mean±SD, n=3). *P<0.0001; #P<0.001.

FIG. 3A-L shows inhibition of DGAT1 causes mitochondrial damage, ROS production and GBM cell apoptosis. (A, B) Transmission electron microscopy (TEM) analysis of the mitochondrial morphology in GBM cells (U251) treated with the DGAT1 inhibitor A-922500 (20 μg/ml) or DGAT2 inhibitor PF-06424439 (20 μg/ml) for 24 hr and shown in a or infected with shRNA-expressing lentivirus against DGAT1 for 48 hr and shown in b. Scale bar, 500 nm. Mitochondria are indicated by red arrows. Mitochondria length and loss of cristae (%) were quantified by ImageJ over 100 mitochondria in each group (mean±SEM). Statistical significance was determined by one-way ANOVA. * P<0.0001. (C, D) Representative fluorescence images of LDs (green) and mitochondria (red) in U251 cells treated with DGAT1 or DGAT2 inhibitor and shown in c or shRNA in (D) as in panels (A, B). LDs were stained with BODIPY 493/503 (green) and mitochondria were stained with MitoTracker Red. Nuclei were stained with Hoechst 33342 (blue). Scale bar, m. The percentage of cells with tubular or fragmented mitochondria was quantified by ImageJ in more than 100 cells (mean±SEM). Statistical significance was determined by one-way ANOVA. * P<0.001. (E, F) Analysis of mitochondrial activity by measuring oxygen consumption rate (OCR) in U251 cells treated with DGAT1/DGAT2 inhibitors or shRNA as in panels (A, B). Lower panels show the quantification of relative basal and maximal OCR change (details please see Methods). Data represent the mean±SD from 3 independent experiments. Basal OCR was determined as OCR before oligomycin subtract OCR after rotenone addition, and maximal OCR was determined as OCR after FCCP subtract OCR after rotenone addition. Statistical significance was determined by one-way ANOVA. * P<0.0001. (G, H) Representative fluorescence images of reactive oxygen species (ROS) production and mitochondria (green) in U251 cells treated with DGAT1/DGAT2 inhibitors or shRNA as in panels (A, B). ROS were detected with CellROX Deep Red, and mitochondria were stained with MitoTracker Green. Nuclei were stained with Hoechst 33342 (blue). Scale bar, 10 m. ROS levels were quantified by ImageJ in more than 100 cells (mean±SD). Statistical significance was determined by one-way ANOVA. * P<0.0001. (I, J) Representative fluorescence images of ROS levels (red) detected by CellROX Deep Red in U251 cells treated with the DGAT1 inhibitor or shRNA as in panels a and b in the presence or absence of N-acetyl-cysteine (NAC, 1 mM). Nuclei were stained with Hoechst 33342 (blue). Scale bar, 10 m. ROS levels were quantified by ImageJ in more than 100 cells (mean±SD) (right upper panels). Live and dead cells were counted by trypan blue staining after treatment for 3 days (mean±SD, n=3) (right bottom panels). Statistical significance was determined by one-way ANOVA. * P<0.0001. (K, L) Western blotting analysis of cytochrome c in cytosol (Cyto) and mitochondria (Mito) and apoptosis markers in GBM cells treated with the DGAT1 inhibitor (20 μg/ml) or DGAT2 inhibitor PF-06424439 (20 μg/ml) for 24 hr (K) or lentivirus-mediated shRNA against DGAT1 for 72 hr (L).

FIG. 4A-H shows genetic inhibition of DGAT1 significantly alters lipid homeostasis and dramatically elevates acylcarnitine and acetyl-CoA levels in GBM cells. (A) Heatmaps of representative lipids in U251 cells analyzed by lipidomics after knockdown of DGAT1 using lentivirus-mediated shRNA for 60 hr versus control cells that were infected with scramble shRNA. The lipid levels in DGAT1 knockdown cells were normalized to control cells. (B-G) Levels of representative individual lipid species in different lipid categories in U251 cells after knockdown of DGAT1 vs. control cells. The data represent the mean±SEM (n=5). Statistical significance was determined by one-way ANOVA. *P<0.0001; #P<0.001; % P<0.01; $P<0.05 in comparison to control cells. Triglycerides (TG), Diacylglycerol (DAG), Monoacylglycerol (MAG), Ceramide (CER), Sphingomyelin (SM), Phosphatidylcholine (PC), Phosphatidylethanolamine (PE), Phosphatidylserine (PS), Phosphatidylinositol (PI), Phosphatidylglycerol (PG), Phosphatidic acid (PA), Lysophosphatidylcholine (LPC), Lysophosphatidylethanolamine (LPE), Lysophosphatidylserine (LPS), Lysophosphatidylinositol (LPI), Lysophosphatidylglycerol (LPG), Lysophosphatidic acid (LPA), Acyl-carnitine (AC). (H) Diagram summarizing the alteration in lipid profiling of the fatty acid metabolism in GBM cells after knockdown of DGAT1. FFA, free fatty acids; G3P, glycerol-3-phosphate.

FIG. 5A-K shows acylcarnitine increase results in ROS production, mitochondria impairment and GBM cell apoptosis, which are associated with enhanced CPT1A expression induced by DGAT1 inhibition. (A) Total acylcarnitine levels in U251 cells after DGAT1 knockdown vs. control cells analyzed by lipidomics (mean±SEM, n=5). Statistical significance was determined by Student's t-test. * P<0.0001. (B) Representative fluorescence images of mitochondria (red) stained with MitoTracker Red in U251 cells treated with different acyl-carnitines (20 μM) for 24 hr. Nuclei were stained with Hoechst 33342. Scale bar, 10 m. (C) Analysis of the mitochondrial activity by measuring the OCR in U251 cells treated with different acylcarnitines (20 μM) for 24 hr. Right panels show the relative levels of basal and maximal OCR in treated cells in comparison to control cells (mean±SD, n=3). Statistical significance was determined by one-way ANOVA. * P<0.0001. (D) Western blot analysis of apoptosis markers in multiple GBM cells treated with different acylcarnitines (20 μM) for 24 hr. (E) Fluorescence image analysis of ROS levels detected by CellROX Deep Red staining in U251 cells treated with different acyl-carnitines (20 μM) for 24 hours in the presence or absence of NAC (1 mM). Nuclei were stained with Hoechst 33342. Scale bar, 10 m. Relative ROS levels in each treatment group were quantified by ImageJ in more than 100 cells (mean±SEM) (right upper panels). Cell death percentage with/without NAC was calculated after 2 days of treatment by trypan blue staining (mean±SD, n=3) (right bottom panels). Statistical significance was determined by one-way ANOVA. * P<0.001. (F-I) Western blot shown in (F) and (G) or IF shown in (H) and (I) analysis of CPT1A expression in U251 cells upon DGAT1 shRNA knockdown (48 hr) or pharmacological inhibition (A-922500, 20 μg/ml for 24 hr). CPT1B was also analyzed by western blotting and shown in (F, G). Cytochrome c oxidase subunit 4 (COX4) was stained by IF to show mitochondria and shown in (H, I). CPT1A levels examined by IF were quantified by ImageJ in more than 50 cells (mean±SEM) and shown in (H, I) (right panels). Statistical significance was determined by Student's t-test and shown in (H) or one-way ANOVA and shown in (I). * P<0.0001. (J, K) Fluorescence image analysis of ROS levels (red) detected by CellROX Deep Red staining in U251 cells treated with DGAT1 inhibitor A-922500 (20 μg/ml) for 24 hours and shown in (J) or knockdown of DGAT1 by lentivirus-expressed shRNA for 48 hours and shown in (K) in the presence or absence of the CPT1 inhibitor etomoxir (ETO, 6 μM). Nuclei were stained with Hoechst 33342. Scale bar, 10 μm. Relative ROS levels were quantified by ImageJ in more than 100 cells (mean±SEM) (right upper panels). Statistical significance was determined by one-way ANOVA. * P<0.0001. Cell death percentage (%) in different treatment groups was also quantified (right lower panels) (mean±SD, n=3).

FIG. 6A-H shows genetic inhibition of DGAT1 significantly suppresses tumor growth and prolongs overall survival in GBM-bearing mice. (A-D) The effects of knockdown of DGAT1 or DGAT2 alone or in combination on tumor growth in U87/EGFRvIII cells-derived subcutaneous model (1×10⁶ cells/mouse) are shown in (A, B) or intracranial tumor model in nude mice (1×10⁵ cells/mouse) are shown in (C, D). Tumor growth in mouse brain was analyzed by bioluminescence imaging on day 17 after implantation and shown in (C). Mouse overall survival was assessed by Kaplan-Meier curves and shown in (D). Statistical significance for subcutaneous tumor growth and luminescence intensity was determined by one-way ANOVA, * P<0.0001 for a-c, and for mouse overall survival by Log-rank test (n=6). n.s. not significant. (E-G) The effects of shRNA knockdown of DGAT1 or DGAT2 on tumor growth in primary GBM30-luciferase cells-derived intracranial mouse model (1×10⁵ cells/mouse). Tumor growth in mouse brain was analyzed by bioluminescence imaging on day 14 after implantation (mean±SEM, n=7) and shown in e. Statistical significance was determined by one-way ANOVA. *P<0.001 for comparison with control group. Sections of whole mouse brain taken at day 19 after implantation were stained with H&E to visualize tumor growth and shown in (F). Kaplan-Meier plot was used to analyze mouse overall survival and shown in (G). Statistical significance was determined by Log-rank test. (H) Tumor tissues from GBM30-derived intracranial model indicated in panel G that were taken from the mice at the time of sacrifice were analyzed by IHC to examine DGAT1, CPT1A, cleaved Caspase 3 levels and LD numbers (TIP47 staining by IF). Protein expression levels and LD numbers were quantified by ImageJ and averaged from 5 separate areas in each tumor. The results for protein levels were normalized to control tumors (mean±SEM). Statistical significance was determined by one-way ANOVA. * P<0.01 for comparison with scrambled control group. Scale bars, 40 μm for IHC, 10 μm for IF.

FIG. 7A-C shows pharmacological inhibition of DGAT1 dramatically suppresses GBM tumor growth and induces tumor cell apoptosis. (A-C) The effects of the DGAT1 inhibitor A-900225 (120 mg/kg/day, oral gavage, details in Methods) in U87/EGFRvIII cells (1×10⁶ cells/mouse)- or GBM30-derived subcutaneous models (4×10⁶ cells/mouse), as shown by tumor growth rate in A and final isolated tumor images and weights in B. Tumor tissues were analyzed for LD presence by IF (TIP47 staining) and CPT1A and cleaved Caspase 3 levels by IHC in C. LD numbers and protein levels in tumor tissues were quantified by ImageJ and averaged from 5 separate areas in each tumor. The results for protein levels were normalized to control tumors (mean±SEM). Statistical significance was determined by one-way ANOVA. * P<0.01 for comparison with control group. Scale bars, m for IF, 40 m for IHC.

FIG. 8 shows a schematic model illustrating the function of DGAT1 in regulating lipid homeostasis and the cytotoxic effects resulting from its inhibition in GBM cells. DGAT1 upregulation in GBM promotes excess fatty acid storage into TG/LDs to maintain the homeostasis of fatty acid metabolism and prevent oxidative stress (left panel). Inhibiting DGAT1 disrupts lipid homeostasis, increases ceramide levels and ER stress, promotes CPT1A upregulation and excess fatty acids moving into mitochondria for oxidation that leads to high levels of ROS and mitochondrial damage, together to induce marked GBM cell death (right panel).

FIG. 9A-G shows DGAT1 gene expression is much higher than DGAT2 in tumor tissues from the TCGA GBM database and is inversely correlated with patient overall survival. (A) Western blot analysis of DGAT1 expression in human normal brain, GBM tumors, and GBM cell lines that were infected with control shRNA- or shDGAT1-expressing lentivirus for 48 hr. PDIA1 (protein disulfide isomerase family A, member 1), an ER-resident protein, was used as an internal control. (B) Schematic diagram showing the primer design for detection of DGAT1 and DGAT2 gene expression using qPCR. (C) RT-qPCR analysis of DGAT1 vs. DGAT2 mRNA levels in human GBM patients (n=10) using three separate primers as shown in panel b. The expression was normalized to the average DGAT1 expression. Statistical significance was determined by Student's t-test. *P<0.001 (D) Paired profiles of DGAT1 vs. DGAT2 gene expression in GBM tumor tissues from the TCGA and CGGA databases, and in other cancer tissues from the TCGA database. TPM, transcripts per million; FPKM, fragments per kilobase million; RPKM, reads per kilobase million. (E) DGAT1 (top) vs. DGAT2 (bottom) gene expression pattern in 30 cancer types in the TCGA database from cBioPortal. (F, G) Kaplan-Meier plot of patient survival in GBM subtypes (F) or total patient population (G) based on DGAT1 expression levels in the GBM TCGA RNAseq (f) or Rembrandt (g) databases using optimal cutoff. The cutoff is 9.711 for the proneural subtype, 9.436 for the classical and 9.471 for the mesenchymal subtypes. Rembrandt GBM cutoff is 8.109. Significance was analyzed by Log-rank test.

FIG. 10A-G shows inhibition of DGAT1, but not DGAT2, significantly suppresses TG/LD formation and induces GBM cell death. (A) RT-qPCR analysis of DGAT1 vs. DGAT2 mRNA levels in different types of cancer cell lines using 3 pairs of different primers. The expression was normalized to the average DGAT1 expression. Statistical significance was determined by Student's t-test. *P<0.01. (B) Representative fluorescence imaging of LDs stained with BODIPY 493/503 (green) (upper panels) and TLC analysis of TG levels (lower panels) in GBM cells after treatment with the DGAT1 inhibitor A-922500 (20 μg/ml) or DGAT2 inhibitor PF-06424439 (20 μg/ml) for 24 hr. Nuclei were stained with Hoechst 33342 (blue). Scale bar: 10 m. LDs were quantified by the ImageJ software in more than 30 cells in each treatment group (mean±SEM). TG levels were quantified by the ImageJ software and normalized to control group (mean±SD, n=3). Statistical significance was determined by one-way ANOVA. *P<0.001; #P<0.05. (C) Percentage of dead GBM cells after treatment for 3 days with the DGAT1 or DGAT2 inhibitors as in panel B. Statistical significance was determined by one-way ANOVA. *P<0.0001. (D) Western blotting analysis of DGAT1 expression in GBM cancer cells after infection with shRNA-expressing lentivirus against DGAT1 for 48 hr. PDIA1 was used as an internal control. (E) RT-qPCR analysis of DGAT2 mRNA levels in GBM cells after infection with shRNA-expressing lentivirus against DGAT2 for 48 hr. The expression level was normalized to control cells and shown as mean±SD (n=3). Statistical significance was determined by one-way ANOVA. *P<0.001. (F) Representative fluorescence images of LDs stained with BODIPY 493/503 (green) (upper panels) and TLC analysis of TAG levels (lower panels) in GBM cells after infection with two independent clones of shRNA-expressing lentivirus against DGAT1 or DGAT2 for 48 hr. Nuclei were stained with Hoechst 33342 (blue). Quantification and significance analysis of LDs in different groups were the same as in panel F in FIG. 2. *P<0.0001. TLC analysis of TG levels in GBM cells infected with two independent clones of shRNA-expressing lentivirus against DGAT1 for 48 hr. Quantification and significance analysis of TG in different groups were the same as in panel b in FIG. 2. *P<0.01. (G) Percentage of dead GBM cells after infection with shRNA-expressing lentivirus against DGAT1 at 48, 60, 72, and 96 hr. Dead cells were counted by trypan-blue staining. The significance was determined by one-way ANOVA (mean±SD, n=3). *P<0.0001.

FIG. 11A-K shows inhibition of DGAT1, but not DGAT2, causes severe mitochondria damage and membrane potential reduction in GBM cells. (A, B) Transmission electron microscopy analysis of the mitochondrial structure in U251cells treated with the DGAT1 inhibitor A-922500 (20 μg/ml) or DGAT2 inhibitor PF-06424439 (20 μg/ml) for 24 hr (A) or infected with shRNA-expressing lentivirus against DGAT1 for 48 hr (B). Scale bar, 500 nm. Mitochondria are indicated by arrows. (C) Representative fluorescence images of mitochondria in U251 cells treated with the DGAT1 inhibitor or DGAT2 inhibitor. Mitochondria were stained with MitoTracker Red. LDs were stained with BODIPY 493/503. Nuclei were stained with Hoechst 33342. Scale bar, 10 μm. (D) Representative fluorescence images of mitochondria in GBM cells infected with shRNA-expressing lentivirus against DGAT1 or DGAT2 for 48 hr. Mitochondria were stained with MitoTracker Red. LDs were stained with BODIPY 493/503. Nuclei were stained with Hoechst 33342. Scale bar, 10 μm. (E, F) Representative fluorescence image analysis of mitochondrial membrane potential stained with Rhodamine123 in U251 cells treated with the DGAT1 inhibitor A-922500 (20 μg/ml) for 24 hr or infected with shRNA-expressing lentivirus against DGAT1 for 48 hr. Nuclei were stained with Hoechst 33342. Scale bar, 10 μm. Rhodamine123 staining was quantified by ImageJ software in more than 100 cells. Statistical significance was determined by s t-test for inhibitor treatment or one-way ANOVA for shRNA knockdown. *P<0.0001. (G-I) Analysis of mitochondrial activity by measuring oxygen consumption rate (OCR) and extracellular acidification rate (ECAR) in different GBM cells treated with the DGAT1 inhibitor A-922500 (20 μg/ml) or DGAT2 inhibitor PF-06424439 (20 μg/ml) for 24 hr, or infected with shRNA-expressing lentivirus against DGAT1 or DGAT2 for 48 hr. Data represent the mean±SD from 3 independent experiments.(J, K) Western blot analysis of apoptosis markers in multiple GBM cells treated with DGAT1 inhibitor or DGAT2 inhibitor for 24 hr (j) or lentivirus-mediated shRNA for 72 hr (k).

FIG. 12A-C shows pharmacological inhibition of DGAT1 induces ER stress, while genetic inhibition does not have effects in GBM cells. (A) Western blotting analysis of ER stress protein markers BIP and CHOP expression in GBM cells treated with the DGAT1 inhibitor A-922500 (20 μg/ml) for 24 hr. (B) Percentage of cell death in U251 cells treated with the ER stress pathway PERK inhibitor GSK2656157 or IRE1a inhibitor 4μ8C, alone or in combination with the DGAT1 inhibitor A-922500 (20 μg/ml) for 3 days. No rescue effects were observed with the combination treatment. (C) Western blotting analysis of expression of ER stress protein markers BIP and CHOP in GBM cells infected with shRNA-expressing lentivirus against DGAT1 for 48 hr.

FIG. 13A-C shows blocking the storage of excess free fatty acids upon DGAT1 inhibition dramatically induces cytotoxicity in GBM cells. (A) Representative fluorescence images of LDs in GBM U251 cells cultured in charcoal-stripped FBS media supplemented with the indicated free fatty acids alone or in mixture for 16 hr after DGAT1 shRNA knockdown for 32 hr. LDs were stained with BODIPY 493/503 and nuclei were stained with Hoechst 33342. Scale bar, 10 m. (B) Cell morphology and percentage of cell death in U251 cells cultured in charcoal-stripped FBS media supplemented with the indicated free fatty acids alone or in mixture for 16 hr and 40 hr after DGAT1 shRNA knockdown for 32 hr. Scale bar, 50 m. (C) Representative fluorescence images of ROS stained by CellROX Deep Red in U251 cells cultured in charcoal-stripped FBS media supplemented with the indicated free fatty acids alone or in mixture for 16 hr after DGAT1 shRNA knockdown for 32 hr. Nuclei were stained with Hoechst 33342 (blue). Scale bar, 10 m.

FIG. 14A-E shows genetic inhibition of DGAT1 does not cause free fatty acids accumulation, while it significantly alters the profiles of phospholipids and lysophospholipids. (A) The profiling of free fatty acids in U251 cells after shRNA knockdown (60 hr) of DGAT1 vs. shRNA control cells. (B, C) The levels of different phospholipids (b) or lysophospholipids (c) in U251 cells with DGAT1 knockdown in comparison with control cells. Statistical significance was determined by Student's t-test. *P<0.05. Phosphatidylcholine (PC), Phosphatidylethanolamine (PE), Phosphatidylserine (PS), Phosphatidylinositol (PI), Phosphatidylglycerol (PG), Phosphatidic acid (PA), Lysophosphatidylcholine (LPC), Lysophosphatidylethanolamine (LPE), Lysophosphatidylserine (LPS), Lysophosphatidylinositol (LPI), Lysophosphatidylglycerol (LPG), Lysophosphatidic acid (LPA). (D, E) Comparison of the components of fatty acids with saturated and unsaturated species in membrane structural lipids, i.e., glycerolphospholipids (PS, PG, PI, PC, PE and PA) and lysophospholipids (LPS, LPG, LPI, LPC and LPE) in U251 cells after shRNA knockdown of DGAT1 vs. control cells. SFA, saturated fatty acid; MUFA, monounsaturated fatty acid; PUFA, polyunsaturated fatty acids. * P<0.0001; #P<0.001; % P<0.01; $P<0.05.

FIG. 15A-E shows supplementing GBM cells with acylcarnitines results in mitochondria damage and marked GBM cells death, but in only minor effects on ER stress. (A) Representative fluorescence images of mitochondria (red) stained with MitoTracker Red in GBM cells treated with different acylcarnitines (20 μM) for 24 hr. Nuclei were stained with Hoechst 33342 (blue). Scale bar, 10 m. (B) Analysis of ECAR by Seahorse in U251 cells treated with different acylcarnitines (20 μM) for 24 hr. (C) Western blot analysis of cytochrome c levels in the cytosol and mitochondria in U251 and GBM30 cells after supplementation with an acylcarnitine mixture (C16:1-, C18:0- and C18:1-carnitines; 20 μM) for 24 hr. (D) Percentage of dead GBM cells after supplementation with C16:1-, C18:0-, or C18:1-carnitine (20 μM) for 48 hr. Statistical significance was determined by one-way ANOVA. *P<0.0001 as compared with untreated control cells. (E) Western blotting analysis of ER stress protein markers BIP and CHOP expression in multiple GBM cells supplemented with different acylcarnitines as in panel A for 24 hr.

FIG. 16A-B shows reducing ceramide synthesis does not rescue cell death induced by DGAT1 inhibition. Left panel (A) shows the schematic diagram for ceramide de novo synthesis regulated by serine palmitoyltransferase (SPT). Myriocin is a specific SPT inhibitor. Right panel (B) shows the percentage of cell death in U251 cells treated with different doses of myriocin (0, 0.5, 1, 5, 10, 20 μM) in combination with the DGAT1 inhibitor A922500 (20 μg/ml) for 3 days.

FIG. 17A-B shows no obvious toxic effects in kidney, liver and spleen or weight loss are observed in DGAT1 inhibitor A922500-treated mice. (A) The H&E staining of the liver, kidney and spleen of mice treated with the DGAT1 inhibitor A922500 (120 mg/kg/day, oral gavage) for 13 days. Scale bar, 50 μm. (B) The body weight of mice treated with vehicle or DGAT1 inhibitor A-922500 (120 mg/kg/day, oral gavage) for 13 days. The mice with DGAT1i treatment lost 8% weight at day 13 treatment compared with the mice weight at day 1.

FIG. 18 shows DGAT1 and DGAT2 expression in different cancers.

FIG. 19A-O shows DGAT1 and DGAT2 expression in different cell types. Breast: MCF7; Prostate: PC3; Colorectal: HCT116; Pancreas: MP2, PANC-1; Melanoma: M229; Skin: a431; Thyroid: 8505C and BCPAP; Bladder: HTB5, CRL1749.

FIG. 20A-C shows DGAT1 inhibitor LCQ908 with 1% FBS, day 3, cell proliferation.

FIG. 21 shows cell death of DGAT1 and DGAT2 inhibition combination in liver cancer.

FIG. 22A-D shows MHCC97L DGAT1 and DGAT2 inhibition combination (experiment 1). MHCC97L 6×10⁴ 12 well plate, treated at 24 hours with 1% FBS DMEM with DGAT1 and DGAT2 inhibitors. Picture taken on day 4.

FIG. 23A-D shows MHCC97L DGAT1 and DGAT2 inhibition combination (experiment 2). MHCC97L 6×10⁴ 12 well plate, treated at 24 hours with 1% FBS DMEM with DGAT1 and DGAT2 inhibitors. Picture taken on day 4.

FIG. 24A-D shows HEPG2 DGAT1 and DGAT2 inhibition combination (experiment 1). HEPG2 6×10⁴ 12 well plate, treated at 24 hours with 1% FBS DMEM with DGAT1 and DGAT2 inhibitors. Picture taken on day 4.

FIG. 25A-D shows HEPG2 DGAT1 and DGAT2 inhibition combination (experiment 2). HeG2 8×10⁴ 12 well plate, treated at 24 hours with 1% FBS DMEM with DGAT1 and DGAT2 inhibitors. Picture taken on day 4.

FIG. 26A-D shows Huh7 DGAT1 and DGAT2 inhibition combination (experiment 1). Huh7 4×10⁴ 12-well plate, treated at 24 hours with 1% FBS DMEM with DGAT1 and DGAT2 inhibitors. Picture taken on day 4.

FIG. 27A-D shows Huh7 DGAT1 and DGAT2 inhibition combination (experiment 2). Huh7 4×10⁴ 12-well plate, treated at 24 hours with 1% FBS DMEM with DGAT1 and DGAT2 inhibitors. Picture taken on day 4.

FIG. 28A-D shows Hep3B DGAT1 and DGAT2 inhibition combination (experiment 1). Hep3B 6×10⁴ 12-well plate, treated at 24 hours with 1% FBS DMEM with DGAT1 and DGAT2 inhibitors. Picture taken on day 4.

FIG. 29A-D shows Hep3B DGAT1 and DGAT2 inhibition combination (experiment 2). Huh7 6×10⁴ 12-well plate, treated at 24 hours with 1% FBS DMEM with DGAT1 and DGAT2 inhibitors. Picture taken on day 4.

Additional advantages will be set forth in part in the description which follows, and in part will be obvious from the description. The advantages will be realized and attained by means of the elements and combinations particularly pointed out in the appended claims. It is to be understood that both the foregoing general description and the following detailed description are exemplary and explanatory only and are not restrictive, as claimed.

### DETAILED DESCRIPTION

The disclosed embodiments can be understood more readily by reference to the following detailed description and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in practice or testing, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the disclosed embodiments are not entitled to antedate such publication by virtue of prior invention.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, “noncancerous cells” can refer to cells that are normal or cells that do not exhibit any metabolic or physiological characteristics associated with cancer. For example, noncancerous cells are healthy and normal cells.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a patient. A patient refers to a subject afflicted with a disease or disorder, such as, for example, cancer and/or aberrant cell growth. The term “patient” includes human and veterinary subjects. In an aspect, the subject has been diagnosed with a need for treatment for cancer and/or aberrant cell growth.

The terms “treating”, “treatment”, “therapy”, and “therapeutic treatment” as used herein refer to curative therapy, prophylactic therapy, or preventative therapy. As used herein, the terms refer to the medical management of a subject or a patient with the intent to cure, ameliorate, stabilize, or prevent a disease, pathological condition, or disorder, such as, for example, cancer or a tumor. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the disease, pathological condition, or disorder is cancer, such as, for example, liver cancer. In an aspect, cancer can be any cancer known to the art.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. For example, in an aspect, preventing can refer to the preventing of replication of cancer cells or the preventing of metastasis of cancer cells.

As used herein, the term “diagnosed” means having been subjected to a physical examination by a person of skill, for example, a physician or a researcher, and found to have a condition that can be diagnosed or treated by compositions or methods disclosed herein. For example, “diagnosed with cancer” means having been subjected to a physical examination by a person of skill, for example, a physician or a researcher, and found to have a condition that can be diagnosed or treated by a compound or composition that alleviates or ameliorates cancer and/or aberrant cell growth.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to cancer and/or aberrant cell growth) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a peptide, or a composition, or pharmaceutical preparation to a subject. Such methods are well known to those skilled in the art and include, but are not limited to, intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed composition or peptide or pharmaceutical preparation and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compare to a control or a sham or an untreated sample).

As used herein, the terms “effective amount” and “amount effective” refer to an amount that is sufficient to achieve the desired result or to have an effect on an undesired condition. For example, in an aspect, an effective amount of a peptide is an amount that kills and/or inhibits the growth of cells without causing extraneous damage to surrounding non-cancerous cells. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

By “modulate” is meant to alter, by increase or decrease. As used herein, a “modulator” can mean a composition that can either increase or decrease the expression level or activity level of a gene or gene product such as a peptide. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition.

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The term “agent” as used herein means any compound or substance such as, but not limited to, a small molecule, nucleic acid, polypeptide, peptide, drug, ion, etc. An “agent” can be any chemical, entity, or moiety, including, without limitation, synthetic and naturally-occurring proteinaceous and non-proteinaceous entities. In some embodiments, an agent is a nucleic acid, a nucleic acid analogue, a protein, an antibody, a peptide, an aptamer, an oligomer of nucleic acids, an amino acid, or a carbohydrate, and includes, without limitation, proteins, oligonucleotides, ribozymes, DNAzymes, glycoproteins, siRNAs, lipoproteins, aptamers, and modifications and combinations thereof etc. In certain embodiments, as described herein, agents are small molecules having a chemical moiety. Compounds can be known to have a desired activity and/or property, e.g., modulate DGAT1 and DGAT2 activity, or can be selected from a library of diverse compounds, using, for example, the screening methods described herein.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “enhance” as used herein means to improve the quality, amount, or strength of a phenomenon, especially a biological response.

### Methods and Compositions

Disclosed are the components to be used to prepare a composition of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

All patents, patent applications, and other scientific or technical writings referred to anywhere herein are incorporated by reference in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed can be resorted to by those skilled in the art, and that such modifications and variations are considered to be within the scope of this invention as defined by the description and the appended claims.

### General

Lipid droplets (LDs), the specific lipid-storage organelles, are prevalent in glioblastoma (GBM), the most lethal primary brain tumor. It was found that LDs are also formed in a variety of cancer cell lines, including liver, lung, melanoma, breast, ovarian, prostate and pancreas cancer cells.

Nevertheless, the role of LDs in cancer cells has remained largely unknown until the present invention. LDs are commonly formed in adipose tissues to store excess fatty acids in human body. LDs store large quantities of triglycerides (TG) and cholesteryl esters, which are surrounded by a phospholipid monolayer that integrates various proteins. The final and committed step for TG synthesis is regulated by two diacylglycerol-acyltransferases, DGAT1 and DGAT2. These enzymes, which are transmembrane proteins residing in the endoplasmic reticulum (ER), catalyze the esterification of fatty acid-CoA with diacylglycerol (DAG) to form TG, which are assembled and bud from the ER into the cytosol to form LDs.

Through large human TCGA cancer patient database analysis, it was found that DGAT1 is highly expressed in Adenoid cystic carcinoma, AMP, bladder, cervical, cholangiocarcinoma, breast, colorectal, glioma, GBM, diffuse large B-cell lymphoma, head & neck, liver, lung, melanoma, mesothelioma, ovarian, pheochromocytoma and paraganglioma (PCPG), pancreas, prostate, sarcoma, testicular germ cell, thymoma, thyroid, uterine, uveal melanoma, rental cancers. It was also found that DGAT2 is highly expressed in liver, bladder, breast, head & neck, thyroid cancers, which cancer types express both high DGAT1 and DGAT2. Interestingly, the data showed that pharmacological inhibition of DGAT1 alone is sufficient to kill most types of cancer cells, even though DGAT2 expression is equal or higher than DGAT1 in Melanoma (M299), Ovarian (HeLa), Bladder (HTB5, CRL1749), Pancreas (MP2 and Panc-1), and Thyroid (BCPAP) cancer cells.

Inhibition of DGAT2 has no effect in any cancer cell lines. Unexpectedly, it was found that the combination inhibition of DGAT1 and DGAT2 are required to induce liver cancer cell death, while either single enzyme inhibition is unable to kill liver cancer cells, including HepG2, MHCC97L, Huh7 and Hep3B cells. It was demonstrated that concurrently targeting DGAT1 and DGAT2 is effective and required to inhibit liver cancer growth (Example 1).

To summarize, it was found that the combination of inhibition of DGAT1 and DGAT2 is effective and required to kill liver cancer cells, while single inhibition of either DGAT1 or DGAT2 has no effect. The combination of DGAT1 and DGAT2 inhibitors can also be effective in treating other types of cancer, as well as DGAT1 inhibition-resistant cancers.

Examples of DGAT1 inhibitors are: 1) Pradigastat (also named LCQ908), Norvatis company; and 2) A922500. Examples of two DGAT2 inhibitors are: 1) PF06424439, Pfizer company; and 2) JNJ DGAT2-A. It was found that DGAT1 inhibitor Pradigastat, also named LCQ908 (Norvatis), and A922500 by Abotta, in combination with DGAT2 inhibitor PF06424449 (Pfizer) or JNJ DGAT2-A, is effective to kill liver cancer cells, while each single inhibitor has no ability to induce liver cancer cell death. In contrast, DGAT1 inhibition alone is effective to cancer cell death in various other cancers. Therefore, disclosed herein is a novel and effective approach to target liver cancer via combination inhibition of DGAT1 and DGAT2.

Methods of Treatment

Disclosed herein is a method of treating cancer in a subject in need thereof, the method comprising administering to the subject an inhibitor of DGAT1, and an inhibitor of DGAT2, or a combination of both. The subject can have liver cancer, and can be diagnosed with liver cancer prior to treatment. The subject can be given a combination of a DGAT1 and a DGAT2 inhibitor. The DGAT1 inhibitor can be Pradigastat and/or A922500, T863, AZD-7687, or AZD 3988. The DGAT2 inhibitor can be PF06424439 and/or JNJ DGAT2-A. The DGAT1 and DGAT2 inhibitors can be given simultaneously or sequentially, such as within 24 or 48 hours. In addition to DGAT1 and/or DGAT2 inhibition, the subject can also be treated with a further liver cancer treatment method such as surgery, ablation, embolization, radiation, targeted therapy, chemotherapy, and immunotherapy. The subject can be monitored during treatment and the treatment is adjusted accordingly.

Further disclosed is a method of treating a subject in need of receiving either DGAT1 inhibition, DGAT2 inhibition, or both, the method comprising: a) selecting a subject diagnosed with cancer; b) measuring levels of DGAT1 and DGAT2 in the subject; c) determining whether the subject is in need of DGAT1 inhibition, DGAT2 inhibition, or both; d) selecting those subjects in need of DGAT1 inhibition, DGAT2 inhibition, or both; and e) treating those subjects in need of DGAT1 inhibition, DGAT2 inhibition, or both accordingly.

Disclosed is a method of treating a subject with cancer, the method comprising administering to the subject a DGAT1 inhibitor, such as Pradigastat and/or A922500, T863, AZD-7687, or AZD 3988.

Further disclosed is a method of treating a subject with cancer, the method comprising administering to the subject a DGAT2 inhibitor such as JNJ DGAT2-A.

The terms “inhibit,” “decrease,” and “reduce”, are all used herein generally to mean a decrease by a statistically significant amount. Accordingly, DGAT1 and/or DGAT2 down regulation is considered to be achieved when the activity value of an DGAT1 and/or DGAT2, or a polynucleotide encoding DGAT1 and/or DGAT2 is about at least 10% less, at least 20% less, at least 30% less, at least 40% less, at least 50% less, at least 60% less, at least 70% less, at least 80% less, at least 90% less, at least 95% less, at least 98% less, at least 99% less, up to including 100% or less, i.e., absent, or undetectable, in comparison to a reference or control level in the absence of the inhibitor. In some embodiments of the aspects described herein, the DGAT1 and/or DGAT2 inhibitors inhibit constitutive DGAT1 and/or DGAT2 activity.

The DGAT1 and DGAT2 inhibitors can be given simultaneously or sequentially. For example, the DGAT1 and DGAT2 inhibitors can be given in the same dose, such as injection, pill, cream, etc. Alternatively, the DGAT1 and DGAT2 inhibitors can be given within seconds, minutes, or hours of each other. For example, the DGAT1 and DGAT2 inhibitor can be given 1, 2, 3, 4, 5, 6, 7, 8, 9, 10, 11, 12, 13, 14, 15, 16, 17, 18, 24, 36, 48, or more hours apart. The doses can be given 3, 4, 5, 6, 7, 10, 14, 28, or more days apart, or any amount above, below or between these ranges.

In addition to DGAT1 and/or DGAT2 inhibition, the subject in need of such inhibition can also be treated with a further treatment method such as surgery, ablation, embolization, radiation, targeted therapy, chemotherapy, and immunotherapy. One of skill in the art will appreciate which treatment method is appropriate and how it should be administered. The subject can be monitored during treatment and the treatment can be adjusted accordingly. In addition to DGAT1 and/or DGAT2 inhibition, the subject can also be treated with a further liver cancer treatment method such as surgery, ablation, embolization, radiation, targeted therapy, chemotherapy, and immunotherapy. The subject can be monitored during treatment and the treatment is adjusted accordingly. Also disclosed is a method of administering to the subject one or more additional therapeutic agents for inhibition of DGAT1 and/or DGAT2. For example, the additional therapeutic agent can be administered months, weeks, days, hours or minutes before the DGAT1 and/or DGAT2 inhibitor, or months, weeks days, hours, or minutes after the DGAT1 and/or DGAT2 inhibitor. It can be administered multiple times throughout a course of administration, such as before, during, and after administration with a DGAT1 and/or DGAT2 inhibitor.

1. In an aspect, a disclosed DGAT1 inhibitor such as Pradigastat and/or A922500, T863, AZD-7687, or AZD 3988, and/or a DGAT2 inhibitor such as PF06424439 and/or JNJ DGAT2-A can be administered to a subject repeatedly. In an aspect, a disclosed composition can be administered to a subject at least two times. In an aspect, a disclosed composition can be administered to the subject two or more times. In an aspect, a disclosed composition can be administered at routine or regular intervals. For example, in an aspect, a disclosed composition can be administered to the subject one time per day, or two times per day, or three or more times per day. In an aspect, a disclosed composition can be administered to the subject daily, or one time per week, or two times per week, or three or more times per week, etc. In an aspect, a disclosed composition can be administered to the subject weekly, or every other week, or every third week, or every fourth week, etc. In an aspect, a disclosed composition can be administered to the subject monthly, or every other month, or every third month, or every fourth month, etc. In an aspect, the repeated administration of a disclosed composition occurs over a pre-determined or definite duration of time. In an aspect, the repeated administration of a disclosed composition occurs over an indefinite period of time.

Kits

Also disclosed herein is a kit comprising a DGAT1 inhibitor and a DGAT2 inhibitor. The kit can comprise a means for delivering the DGAT1 and DGAT2 inhibitors to a subject in need thereof. The DGAT1 and DGAT2 inhibitors can be premixed together. The DGAT1 and DGAT2 inhibitors can be provided in containers designed to be premixed before administration to a subject.

Kits for practicing the methods disclosed herein are further provided. By “kit” is intended any manufacture (e.g., a package or a container) comprising at least one compound or composition disclosed herein. The kit may be promoted, distributed, or sold as a unit for performing the methods of the present disclosure. Additionally, the kits may contain a package insert describing the kit and methods for its use. Any or all of the kit reagents may be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, e.g., about 1 to 100 mg/kg; intranasal instillation, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer drugs, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

Pharmaceutical Compositions

Disclosed herein are DGAT1 inhibitors, such as Pradigastat and/or A922500, as well as DGAT2 inhibitors such as PF06424439 and/or DGAT2a in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. P P. Gerbino, Lippincott Williams & Wilkins, Philadelphia, PA. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the compositions, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

In some embodiments, the molecule is administered in a dose equivalent to parenteral administration of about 0.1 ng to about 100 g per kg of body weight, about 10 ng to about 50 g per kg of body weight, about 100 ng to about 1 g per kg of body weight, from about 1 g to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of molecule administered to achieve a therapeutic effective dose is about 0.1 ng, 1 ng, 10 ng, 100 ng, 1 μg, 10 μg, 100 g, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

### EXAMPLES

### Example 1: DGAT1 Upregulation Promotes Glioblastoma Growth by Regulating Lipid Homeostasis and Preventing Oxidative Stress

Lipid metabolism is reprogrammed in malignancies such as glioblastoma (GBM), but how tumor cells maintain lipid homeostasis to prevent potential lipotoxicity remains elusive. Here, we identify that GBM upregulates diacylglycerol-acyltransferase 1 (DGAT1), but not DGAT2, to store excess fatty acids into triglycerides and lipid droplets. Inhibiting DGAT1 disrupted lipid homeostasis and resulted in excessive fatty acids moving into mitochondria for oxidation. Targeting DGAT1 resulted in generation of high levels of reactive oxygen species (ROS), mitochondrial damage, cytochrome c release and apoptosis. Adding N-acetyl-cysteine or inhibiting fatty acids shuttling into mitochondria decreased ROS and cell death induced by DGAT1 inhibition. Xenograft models show that inhibition of DGAT1 blocked lipid droplet formation, induced tumor cell apoptosis, and markedly suppressed GBM tumor growth. Together, it has been demonstrated that DGAT1 upregulation protects GBM from oxidative damage and maintains lipid homeostasis by facilitating storage of excess fatty acids, and targeting DGAT1 is a worthwhile therapeutic approach for GBM.

It was demonstrated that DGAT1, but not DGAT2, is highly expressed in GBM and plays a dominant role in promoting excess fatty acid storage into TG and LDs. It was further shown that DGAT1 plays a critical role in regulating lipid homeostasis and protecting GBM from oxidative damage induced by excess fatty acids. Inhibiting DGAT1 induces dramatic GBM cell apoptosis and reduction of tumor growth in vitro and in vivo. Thus, the previously unrecognized molecular mechanism regulating lipid homeostasis in GBM has been uncovered and provides a new approach for GBM therapy.

Results

GBM Tissues Contain Large Amounts of TG and Express High Levels of DGAT1 that are Associated with Poor Patient Survival

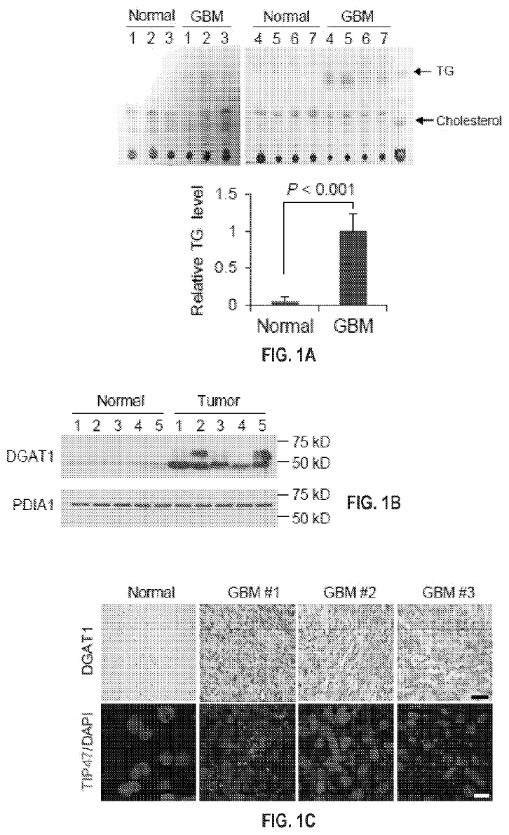

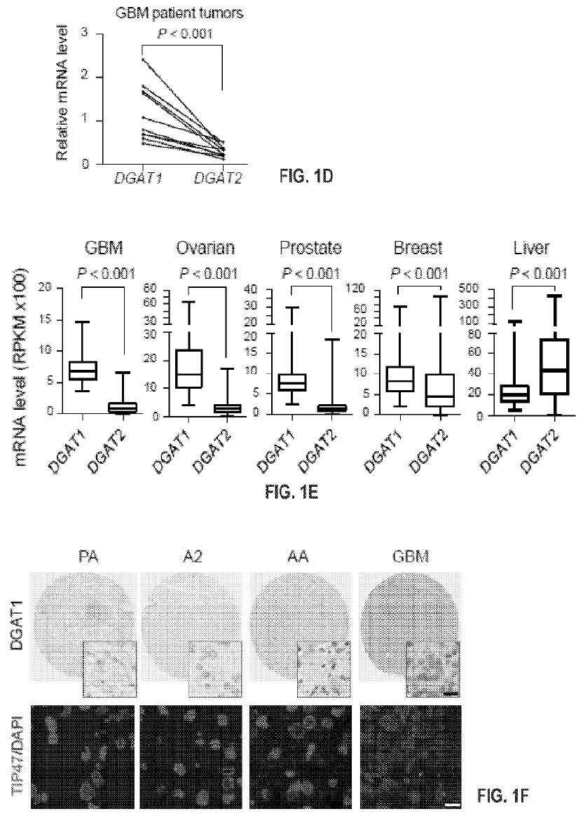

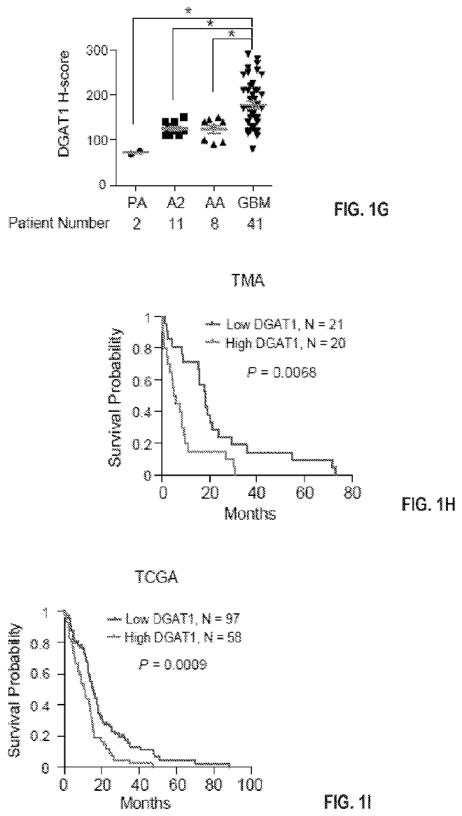

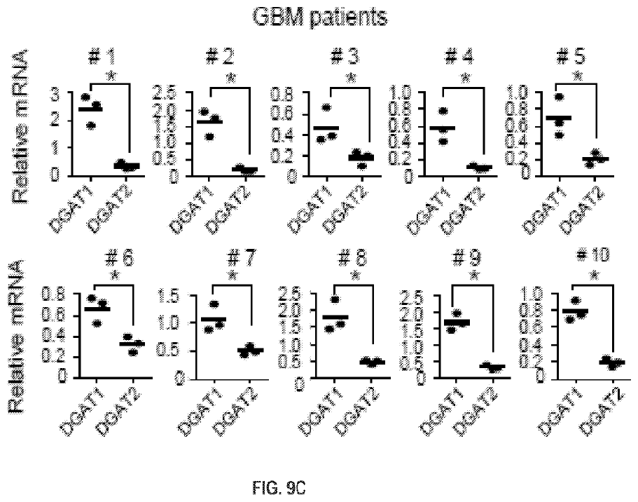

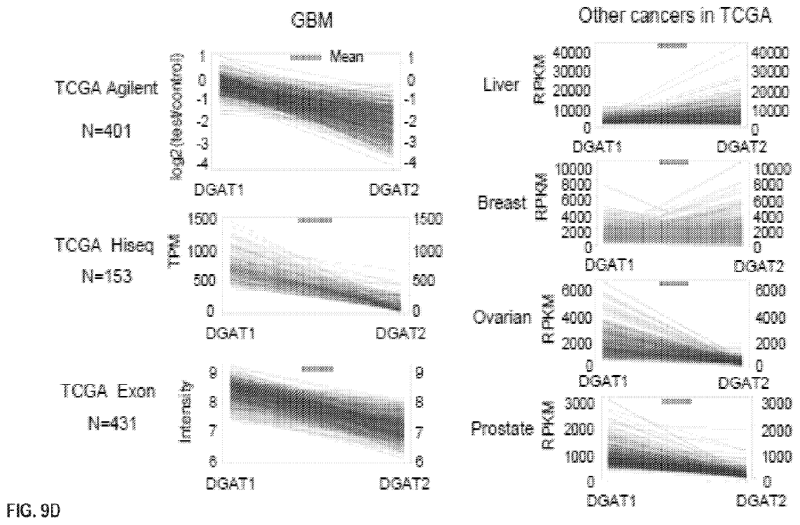

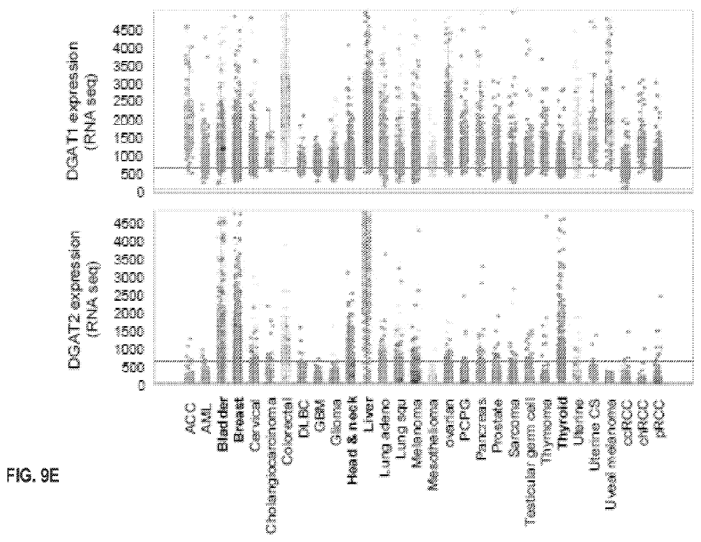

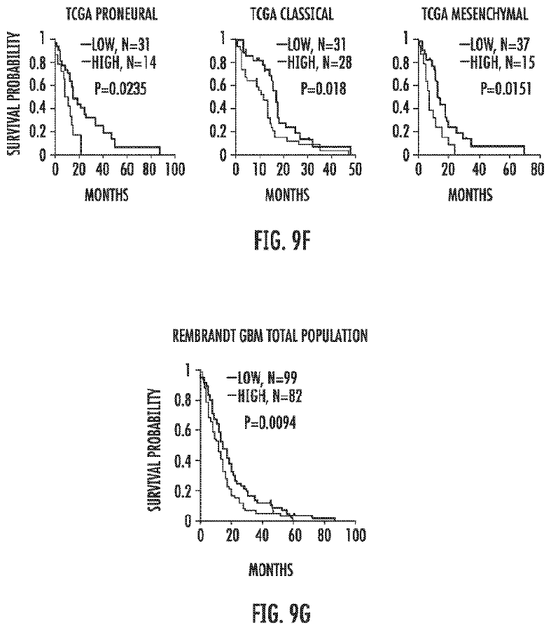

To explore how fatty acid homeostasis is regulated in GBM, it was examined whether TG are formed in patient tumor tissues by using thin layer chromatography (TLC). It was found that GBM tumor tissues contain large amounts of TG as compared with normal brain tissues in which TG could not be detected (FIG. 1a). Western blotting and immunohistochemistry (IHC) analyses showed that DGAT1 is highly expressed in GBM tumor tissues compared to normal brains (FIG. 1b,c, and FIG. 9A), correlating with the prevalence of LDs in tumor tissues, as examined by immunofluorescence (IF) staining of the LD membrane protein, TIP47 (FIG. 1c, lower panels)⁹.

The mRNA levels of DGAT1 vs. DGAT2 were compared in 10 GBM patient specimens by real-time PCR. The data showed that DGAT1 expression was significantly higher than DGAT2 expression in tumor tissues from the same patient (FIG. 1d, FIG. 9b,c). mRNA levels were further examined in GBM tissues from The Cancer Genome Atlas (TCGA) database (Cerami 2012; Gao 2013). The data showed that DGAT1 mRNA expression was much higher than that of DGAT2 in GBM tumor tissues from the database (FIG. 1e and FIG. 9d). TCGA pan-cancer data analysis further showed that high levels of DGAT1 mRNA occurred in ovarian, prostate, breast, liver and many other cancer types (FIG. 1e, and FIG. 9d,e), while high DGAT2 mRNA was only observed in bladder, breast, liver, head & neck and thyroid cancers (FIG. 1e and FIG. 9e), suggesting their differential expression pattern in human cancers.

DGAT1 protein levels were further examined in tumor tissues from patients with grade I-IV astrocytomas using a tissue microarray (TMA) (n=62). IHC staining showed that grade IV GBM tissues contained the highest levels of DGAT1 in comparison with anaplastic astrocytoma (AA, grade III), astrocytoma II (A2) and pilocytic astrocytoma (PA, grade I) (FIG. 1f,g), correlating with the LD prevalence in GBM tissues (FIG. 1f, lower panels). Furthermore, survival analysis showed that high protein levels of DGAT1 in tumor tissues were associated with poor GBM patient survival (FIG. 1h). Accordingly, TCGA gene expression database analyses showed that high levels of DGAT1 mRNA expression in GBM tumor tissues were inversely correlated with patient overall survival (FIG. 1i and FIG. 9f), which was further confirmed by analysis of the Rembrandt gene expression database (FIG. 9g). Together, these data show that DGAT1 can play a critical role in regulating TG/LDs formation and serve as a prognostic marker and molecular target in GBM.

Inhibition of DGAT1, but not DGAT2, Significantly Suppresses TG/LDs Formation and Induces GBM Cell Death

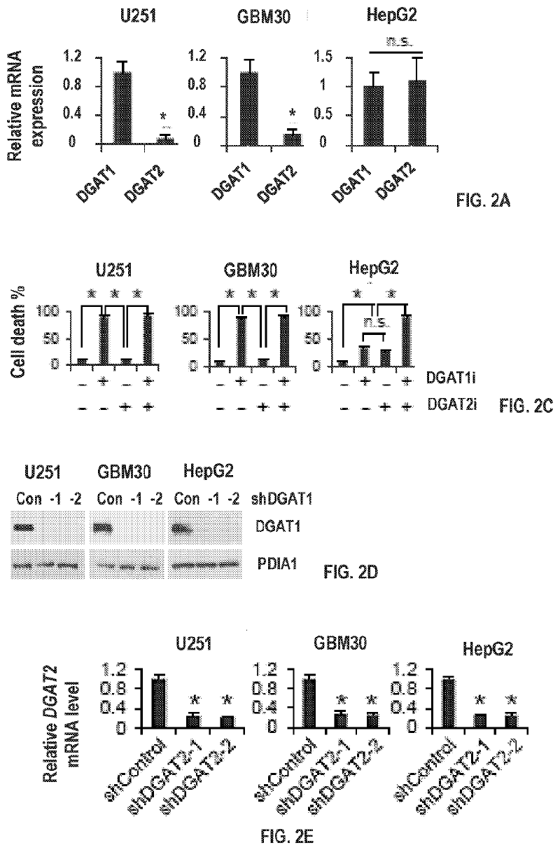

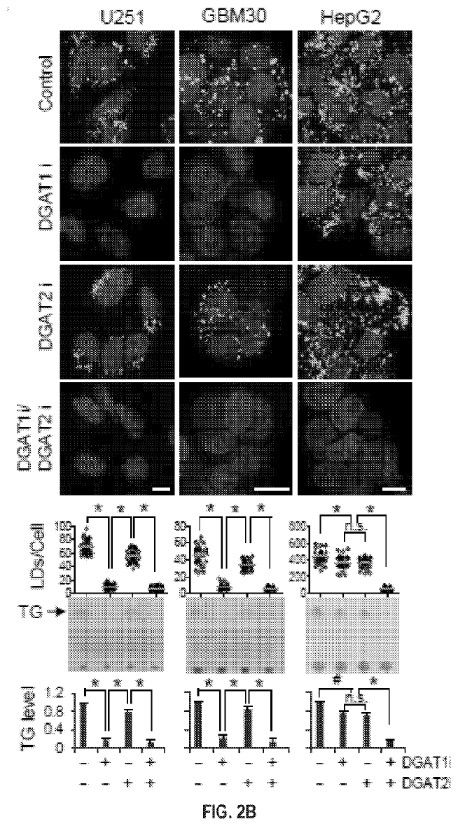

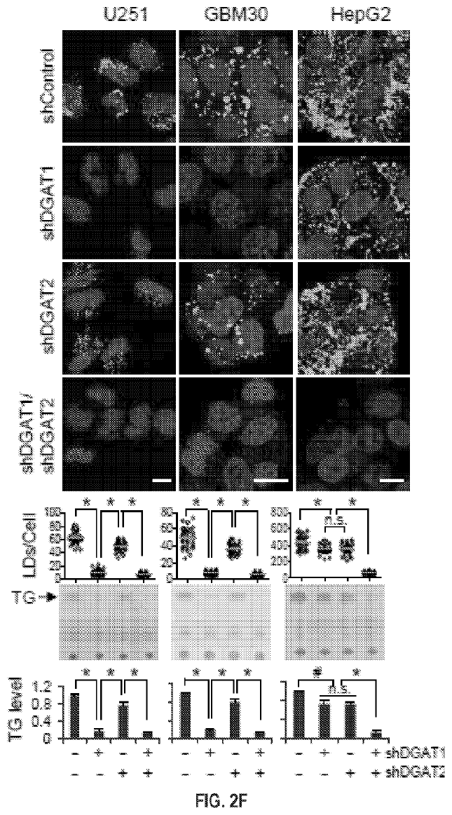

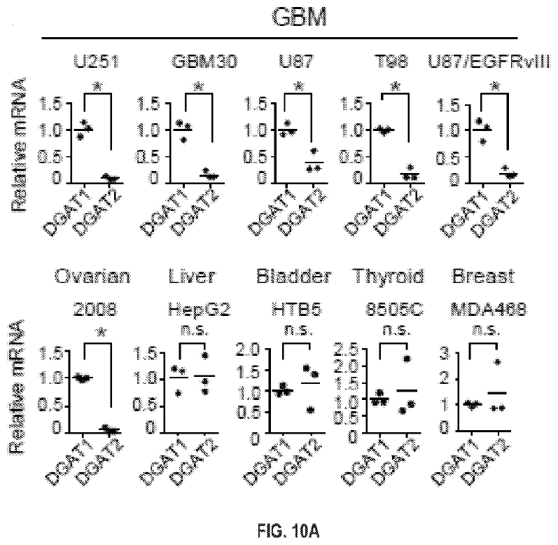

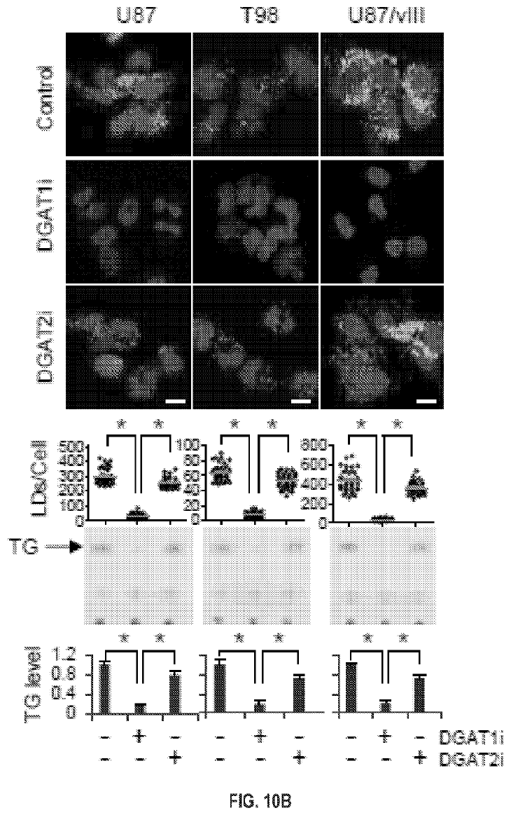

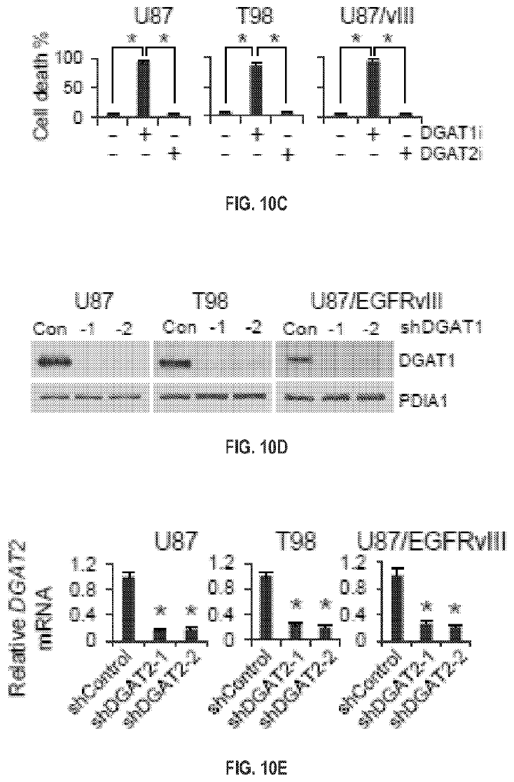

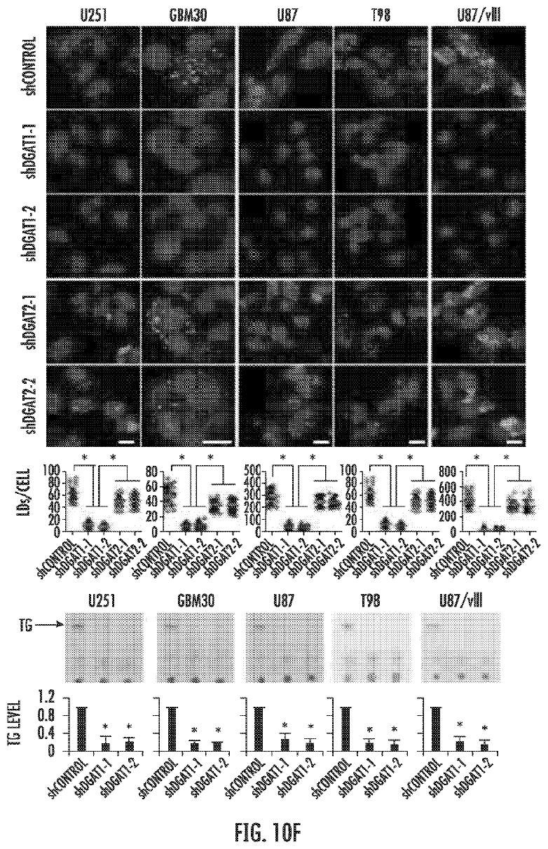

The respective role of DGAT1 and DGAT2 in regulating TG synthesis and LD formation in GBM cells was examined. Real-time PCR showed that DGAT1 expression was also significantly higher than DGAT2 expression in multiple GBM cell lines and patient-derived GBM30 cells^{9, 24} (FIG. 2a, and FIG. 10a), which is consistent with their expression patterns in GBM patient tumor tissues (FIG. 1d,e, and FIG. 9c,d). The pattern of DGAT1 vs. DGAT2 expression in ovarian cancer cell lines was similar as the one in GBM cells, with higher DGAT1 expression than DGAT2 (FIG. 10a). In contrast, the expression of DGAT2 was similar as that of DGAT1 in liver (HepG2), bladder (HTB5), breast (MDA468) and thyroid (8505C) cancer cell lines (FIG. 2a and FIG. 10a). The expression levels of DGAT1 vs. DGAT2 in different types of cancer cell lines are consistent with their expression pattern in large cohorts of cancer patients from the TCGA database (FIG. 1e, and FIG. 9d,e).

GBM cells were treated for 24 hours with inhibitors for DGAT1 (A-922500, 20 g/ml) 25, 26 or DGAT2 (PF-06424439, 20 μg/ml). The data showed that inhibition of DGAT1 dramatically reduced the number of LDs (stained by BODIPY 493/503, upper panels) and levels of TG (analyzed by TLC, lower panels) in all examined GBM cells (FIG. 2b and FIG. 10b). In contrast, pharmacological inhibition of DGAT2 only resulted in a minor reduction of the LDs/TG levels (FIG. 2b and FIG. 10b). Moreover, concurrently inhibiting DGAT1 and DGAT2 did not result in further reduction of the LDs/TG levels compared to DGAT1 inhibition alone (FIG. 2b and FIG. 10b). However, in HepG2 cells, inhibition of DGAT1 or DGAT2 only slightly decreased LDs/TG levels, and inhibition of both enzymes was required to strongly reduce their levels (FIG. 2b). These data suggest that DGAT1 plays a key role in TG/LDs formation in GBM cells, while TG/LDs are controlled by both enzymes in liver cancers. Interestingly, inversely correlated to TG/LDs reduction, pharmacological inhibition of DGAT1 for 3 days greatly induced marked GBM cell death (FIG. 2c, and FIG. 10c), while inhibition of both enzymes was required for cell death of HepG2 cells (FIG. 2c).

A genetic inhibition approach was used to confirm the role of DGAT1 vs. DGAT2 in regulating TG/LDs formation in GBM cells. Consistent with the effects of pharmacological inhibition (FIG. 2b and FIG. 10b), downregulation of DGAT1 for 48 hours via lentivirus-mediated shRNA markedly reduced TG/LDs levels in all examined GBM cells, while DGAT2 knockdown only caused a minor reduction (FIG. 2d-f, and FIG. 10d-f), and no further significant decrease of TG/LDs levels was observed upon combined knockdown of both enzymes (FIG. 2f). In contrast, in HepG2 cells, genetic inhibition of either DGAT1 or DGAT2 only slightly reduced TG/LDs levels, and only the knockdown of both enzymes resulted in dramatic TG/LDs reduction (FIG. 2d-f). Moreover, inversely correlated with TG/LDs reduction (FIG. 2f and FIG. 10f), DGAT1 knockdown led to time-dependent GBM cell death (FIG. 2g and FIG. 10g). In contrast, this effect required knocking down both enzymes in HepG2 cells (FIG. 2g).

Together, these data demonstrate that DGAT1 plays a dominant role in regulating TG synthesis and LDs formation in GBM cells, while DGAT2, expressed at low levels in GBM, plays a minor role in TG/LDs synthesis. Moreover, the data show that targeting DGAT1 to suppress TG/LDs formation induces marked GBM cell death.

DGAT1 Inhibition Results in Mitochondrial Damage, ROS Elevation and GBM Cell Apoptosis

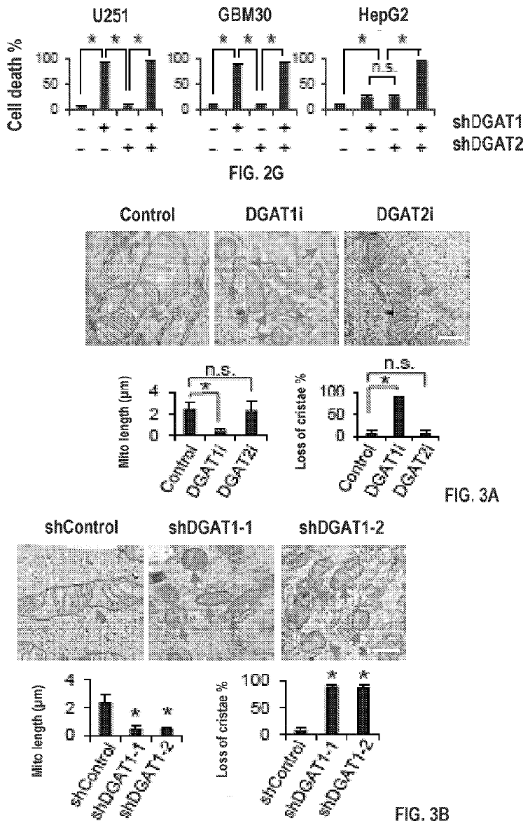

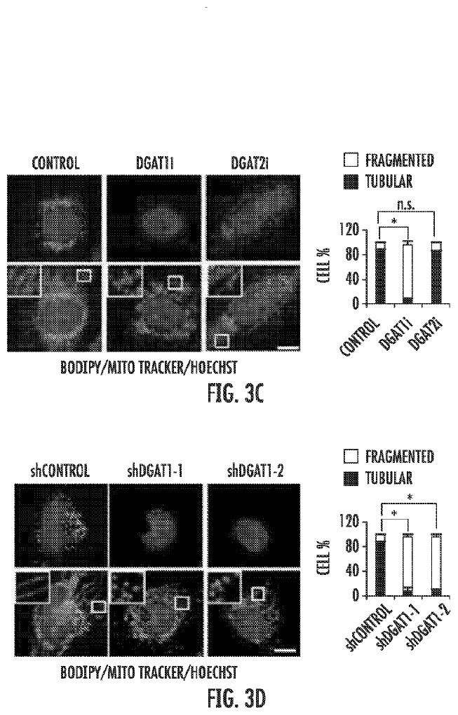

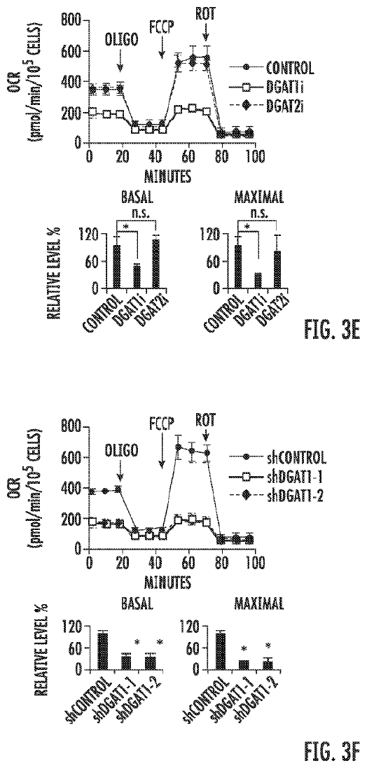

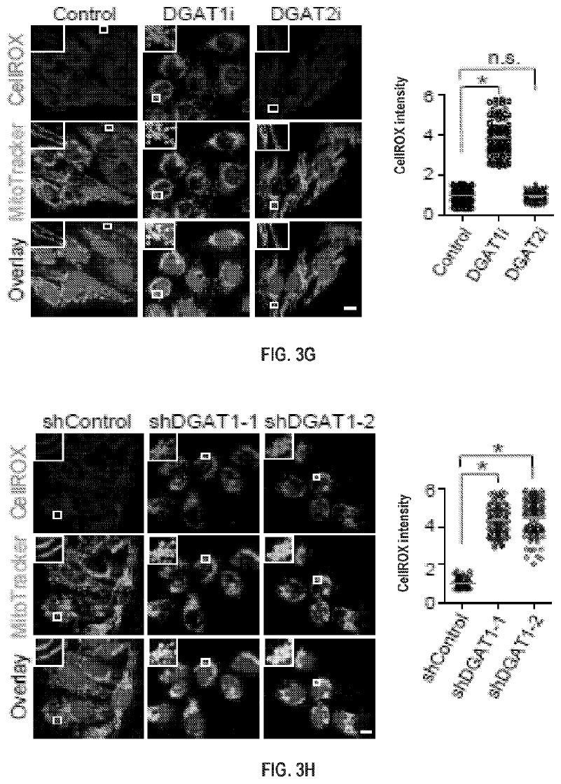

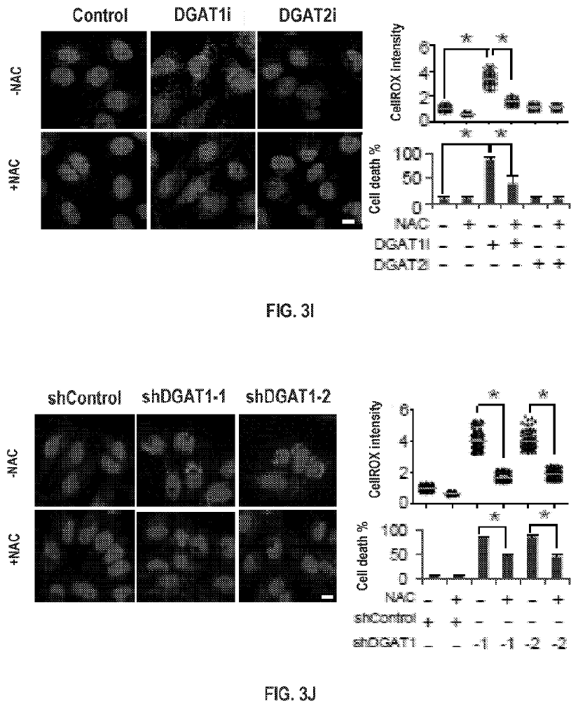

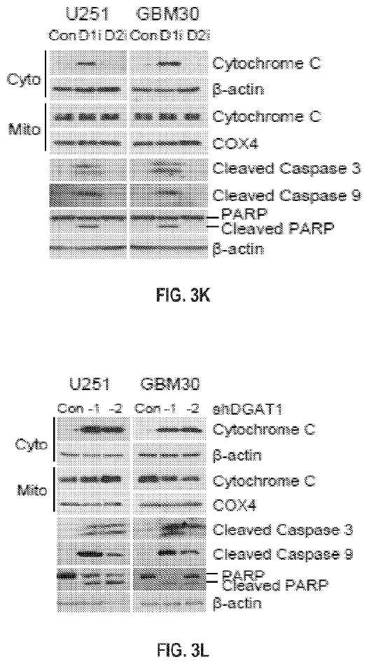

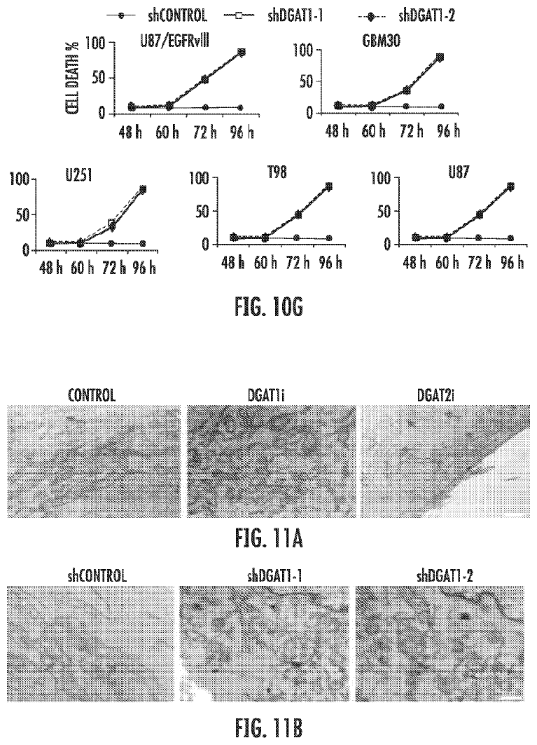

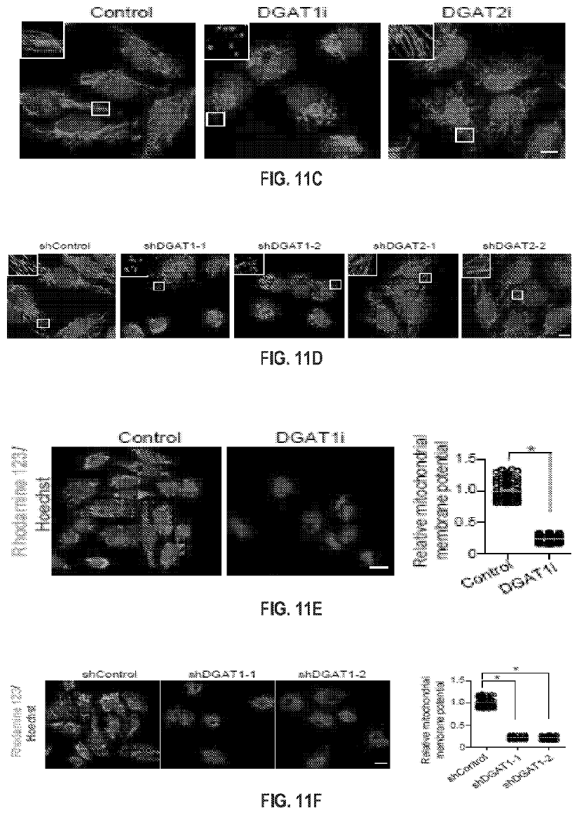

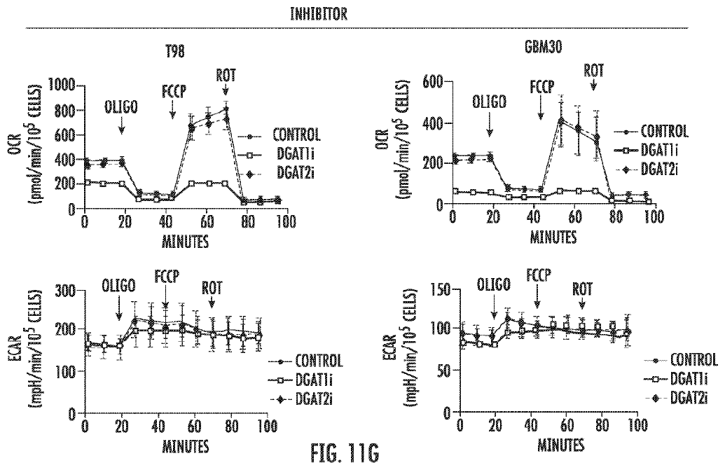

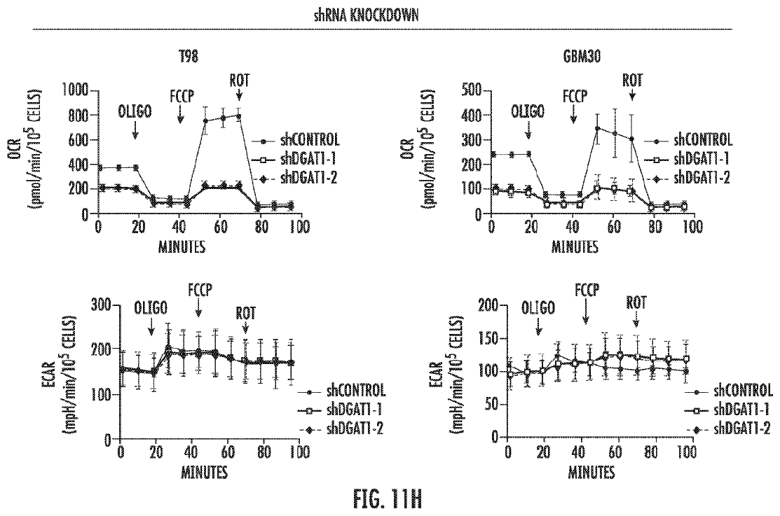

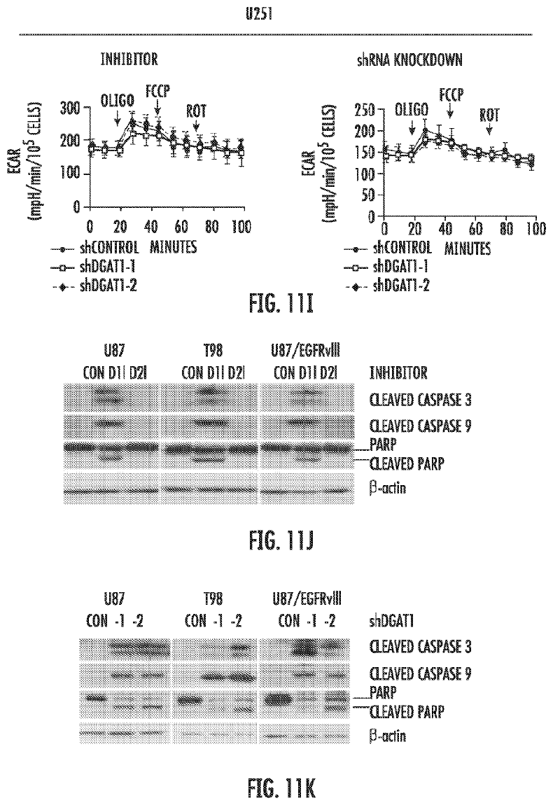

To identify the leading cause of GBM cell death upon DGAT1 inhibition, GBM cellular morphology was examined under this condition by transmission electron microscopy (TEM). Micrographs showed that the structure of mitochondria was severely disrupted upon pharmacological (A922500, 24 hours) or genetic (shRNA, 48 hours) inhibition of DGAT1. Mitochondria became fragmented and round, and lost cristae in comparison with the lengthy tubular shape of mitochondria in control cells (FIG. 3a,b, and FIG. 11a,b). Confocal microscopy was used to examine mitochondria morphology after MitoTracker Red staining. Consistent with the TEM observation, fluorescence imaging showed that the tubular mitochondria (red) observed in control cells became fragmented and round after pharmacological or genetic inhibition of DGAT1, accompanied by the disappearance of LDs (green) (FIG. 3c,d, and FIG. 11c,d). In addition, inhibiting DGAT1 significantly reduced mitochondria membrane potential (FIG. 11e,f). In contrast, there was no obvious damage to mitochondria upon pharmacological or genetic inhibition of DGAT2 (FIG. 3a,c, and FIG. 11c,d).

Next, it was examined whether mitochondria function was impaired upon DGAT1 inhibition by measuring oxygen consumption using a Seahorse instrument (Londe 2018; Cvrljevic 2011). The data showed that both pharmacological (A-922500, 20 μg/ml for 24 hours) and genetic inhibition of DGAT1 (48 hours) markedly reduced the oxygen consumption rate (OCR) in mitochondria compared to control cells, while no reduction was observed upon DGAT2 inhibition by its inhibitor (PF-06424439, 20 μg/ml for 24 hours) (FIG. 3e,f, and FIG. 11g,h). Moreover, the data showed that all treatments did not affect extracellular acidification rate (ECAR) (FIG. 11g-i). These data demonstrate that inhibition of DGAT1 to block TG/LDs formation severely impaired mitochondrial structure and function.

It was then examined whether DGAT1 inhibition led to the production of reactive oxygen species (ROS) in GBM cells. Using CellROX DeepRed staining (Liu 2012) fluorescence imaging showed that ROS (red) were markedly elevated upon pharmacological or genetic inhibition of DGAT1 (FIG. 3g,h). In contrast, inhibition of DGAT2 did not increase ROS production in GBM cells (FIG. 3g). Moreover, the data further showed that the increased ROS production was localized to the mitochondria, as shown by yellow fluorescence in the overlay imaging (FIG. 3g,h), demonstrating that the increased ROS upon DGAT1 inhibition were generated in the mitochondria. It was then examined whether treating cells with the ROS scavenger N-acetyl-cysteine (NAC) (Aruoma 1989) could reduce cell death induced by DGAT1 inhibition. The data showed that NAC significantly decreased both ROS production and cell death caused by DGAT1 inhibition (FIG. 3i,j). Furthermore, western blotting showed that DGAT1 inhibition resulted in high levels of cytochrome c release from the mitochondria into the cytosol (FIG. 3k,l) and strongly induced apoptosis in GBM cells, as demonstrated by the dramatic increase in the cleaved caspase 3, 9 and PARP proteins (FIG. 3k,l, FIG. 11j,k).

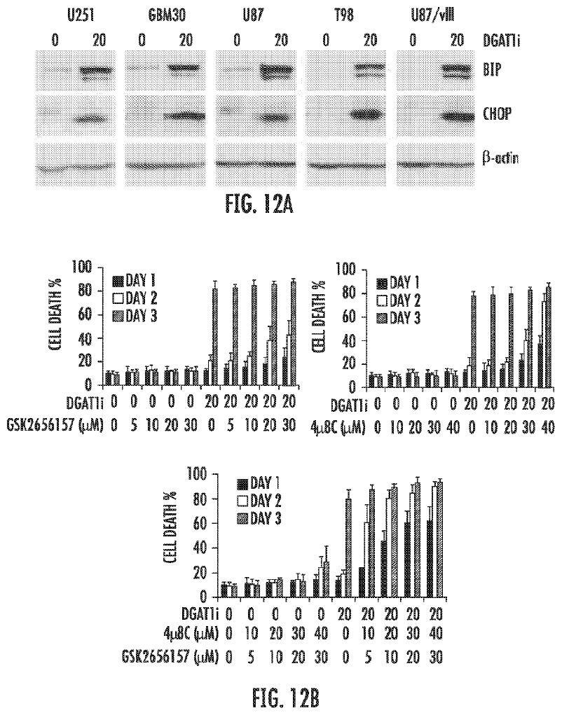

The effects of DGAT1 inhibition on ER stress was further examined and it was found that acute pharmacological inhibition of DGAT1 by its inhibitor A922500 (20 μg/ml, 24 hours) increased the protein levels of BIP and CHOP (FIG. 12a), two key players and biomarkers of ER stress (Lee 2005; Nishitoh 2012). (To examine whether increased ER stress contributes to DGAT1 inhibition-induced GBM cell death, ER stress was inhibited by suppressing PRKR-like endoplasmic reticulum kinase (PERK) with its inhibitor GSK2656157 or inositol-requiring enzyme 1 alpha (IRE1a) inhibitor 4μ8C, two major upstream players in ER stress cascades (Cross 2012; Atkins 2013). These inhibitions, alone or in combination, did not exert any rescue effects on cell death promoted by DGAT1 inhibition (FIG. 12b). Moreover, chronic inhibition of DGAT1 by shRNA knockdown did not induce ER stress, as evident by the unchanged expression of BIP and CHOP (FIG. 12c). In contrast, both pharmacological and genetic inhibition of DGAT1 severely impaired mitochondria (FIG. 3a-e, FIG. 11), increased ROS (FIG. 3g-j), and promoted cytochrome c release and apoptosis (FIG. 3k,j, FIG. 11j,k). Together, these data demonstrate that ROS and mitochondrial damage induced by DGAT1 inhibition are sufficient to trigger GBM cell death.

Inhibiting DGAT1 Disrupts Lipid Homeostasis and Dramatically Increases Acylcarnitine Levels in Association with the Upregulation of CPT1A, Leading to Severe Oxidative Stress that Kills GBM Cells

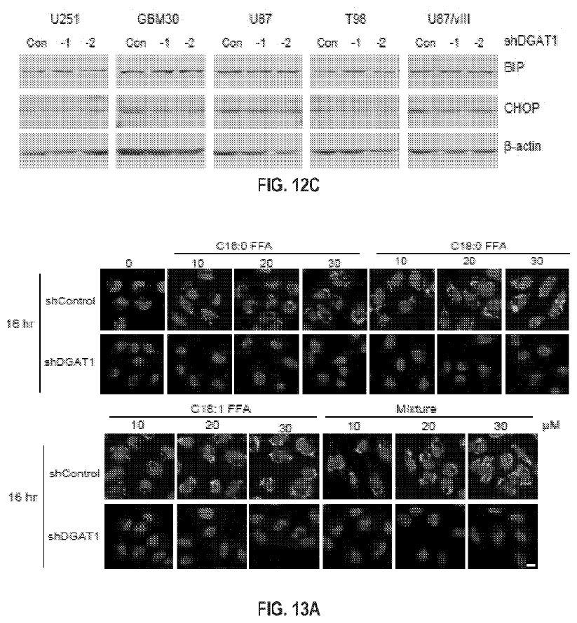

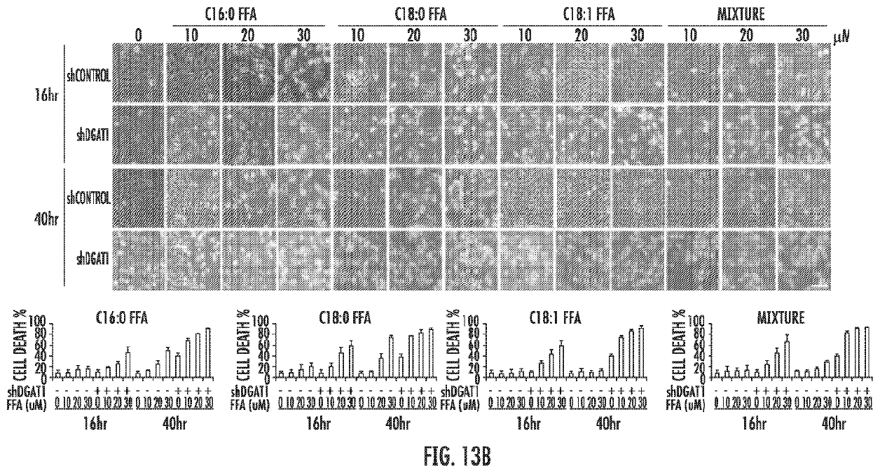

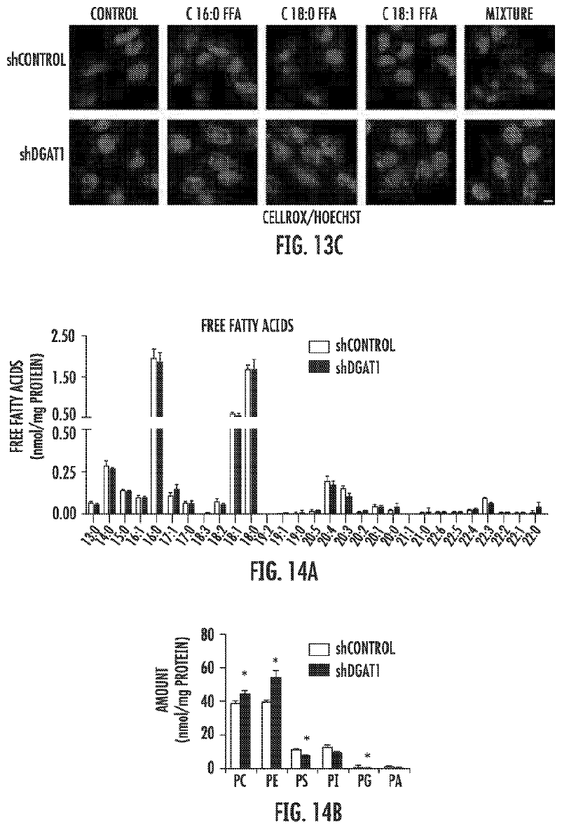

To uncover the underlying mechanism leading to mitochondrial damage and apoptosis upon DGAT1 inhibition, we first checked whether supplementing GBM cells with excess fatty acids could induce severe cytotoxicity when fatty acid storage was blocked. DGAT1 was knocked down using shRNA and then cultured cells in charcoal-stripped FBS media supplemented with individual C16:0, C18:0, C18:1 fatty acids or their mixture at different doses (0, 10, 20 and 30 μM). The data showed that these fatty acids, even at 30 μM, had not induced toxicity in control GBM cells infected with scramble shRNA after 16 hour supplementation (FIG. 13a,b). In contrast, when their storage was blocked in shDGAT1 knockdown cells (FIG. 13a), all fatty acids either alone or in mixture induced marked cell death in a dose- and time-dependent manner (FIG. 13a,b), and resulted in a greater ROS production than either DGAT1 inhibition or fatty acid supplement alone (FIG. 13c). These data demonstrate that unstored excess free fatty acids trigger severe oxidative stress and cell death in GBM.

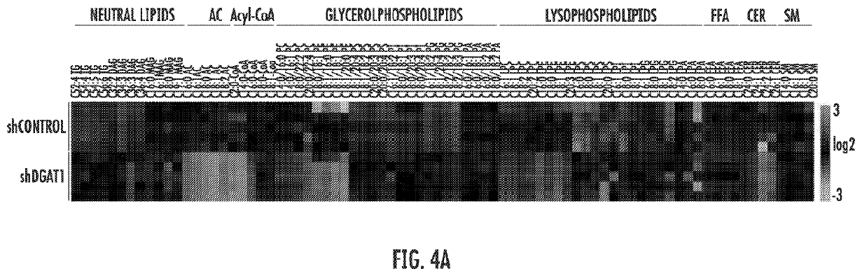

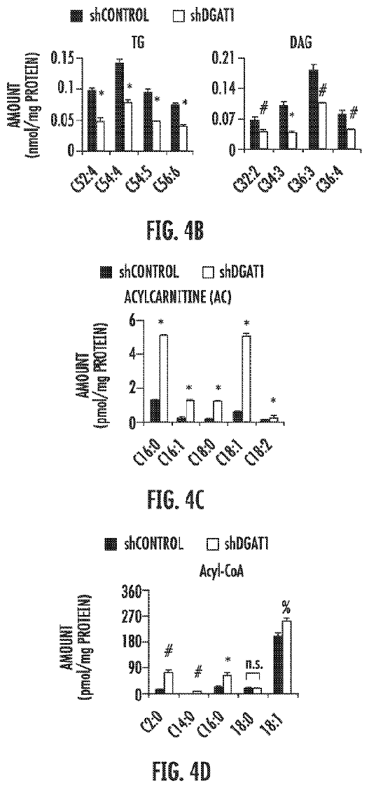

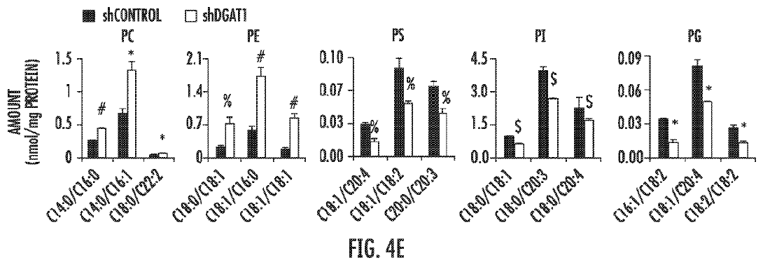

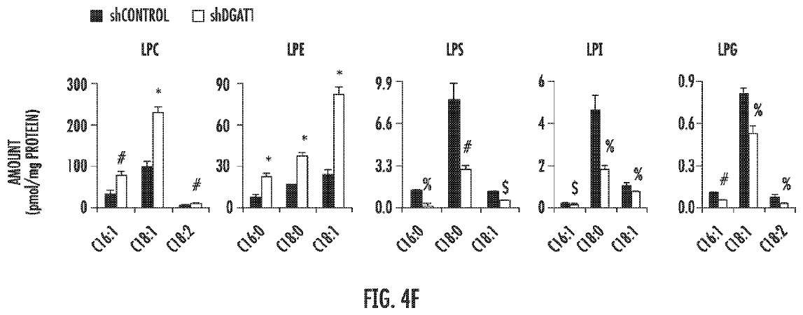

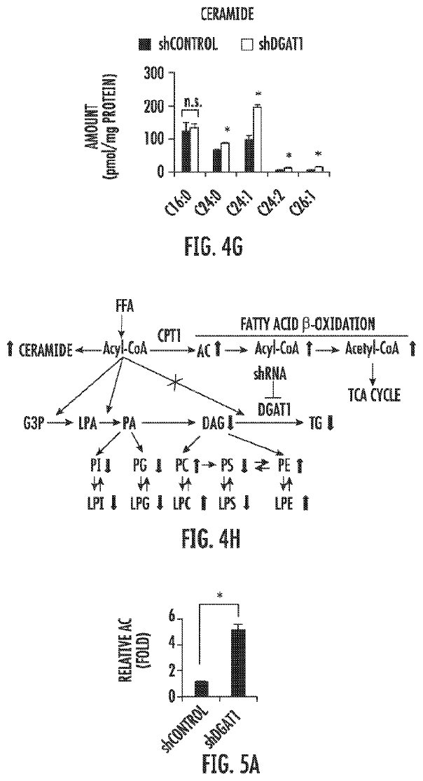

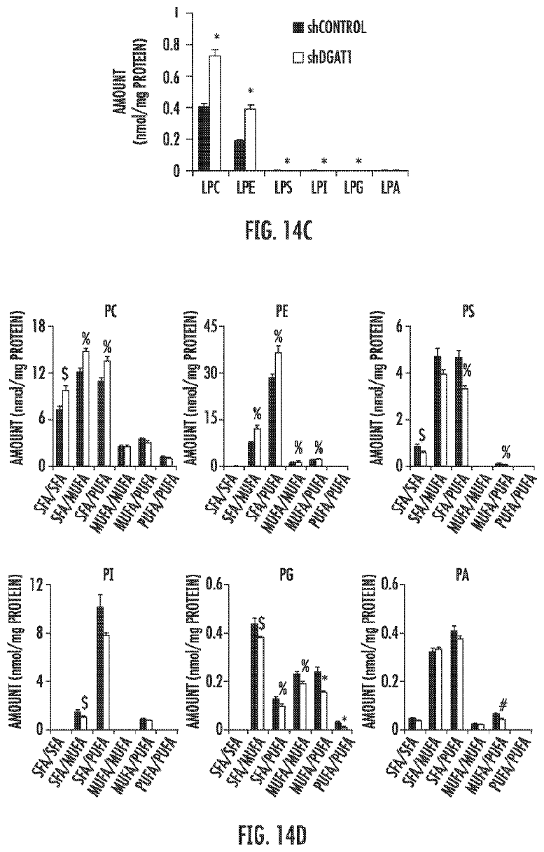

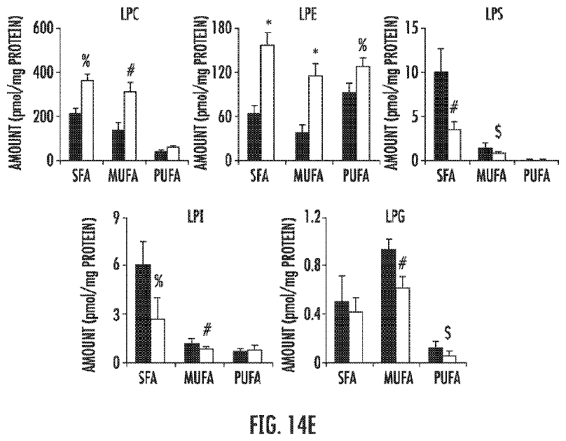

Lipidomics were then performed to examine the levels of free fatty acids in GBM cells upon DGAT1 knockdown compared to control cells infected with scramble shRNA. The data showed that control cells contained a pool of free fatty acids with different lengths and saturation (FIG. 14a). Unexpectedly, no significant increase in free fatty acids in DGAT1 knockdown cells, as compared with control cells (FIG. 4a, and FIG. 14a) was observed, contrary to the original thought that DGAT1 inhibition would cause the accumulation of free fatty acids to induce cytotoxicity and GBM cell death. We then extensively analyzed and compared lipid profiles in GBM cells between DGAT1 knockdown and control groups. It was found that acylcarnitines (AC), which are converted from fatty acids by carnitine palmitoyltransferase 1 (CPT1) and shuttled into mitochondria for oxidation and energy production (Qu 2016; Schreurs 2010), were remarkably increased, along with TG reduction (FIG. 4a-c). Accordingly, the levels of acetyl-CoA (C2:0), the major product of fatty acid breakdown by β-oxidation in mitochondria, were dramatically increased (FIG. 4a,d). Moreover, the major structural lipids, i.e., phosphatidylcholine (PC), phosphatidylethanolamine (PE), lysophosphatidylcholine (LPC), and lysophosphatidylethanolamine (LPE), were significantly increased upon DGAT1 inhibition (FIG. 4a,e,f, and FIG. 14b-e). In contrast, many lipid species in phosphatidylserine (PS), phosphatidylinositol (PI), phosphatidylglycerol (PG), lysophosphatidylserine (LPS), lysophosphatidylinositol (LPI) and lysophosphatidylglycerol (LPG), which are found in low amounts in structural lipids, were downregulated (FIG. 4a,e,f, and FIG. 14b-e). In addition, it was found that ceramides were modestly increased upon DGAT1 inhibition (FIG. 4g). Together, lipidomics analyses show that blocking TG/LDs formation does not induce free fatty acids accumulation, but shifts them to the major structural lipids PC/PE and mitochondria, leading to the disruption of lipid homeostasis and oxidative stress (FIG. 4h).

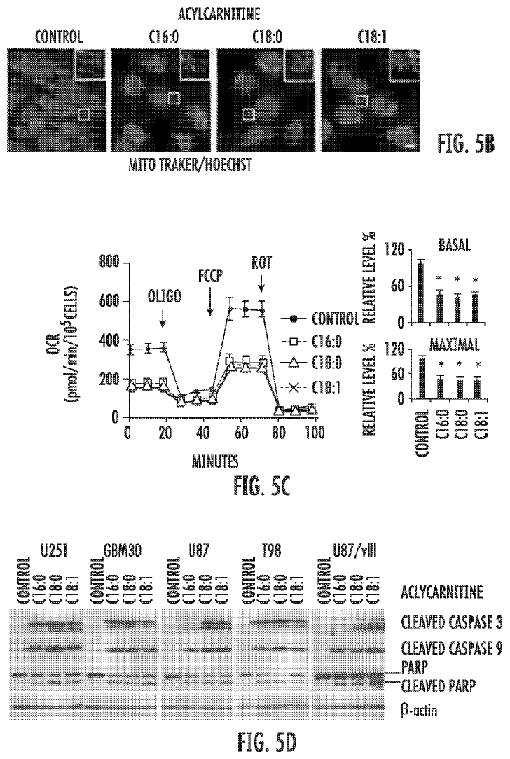

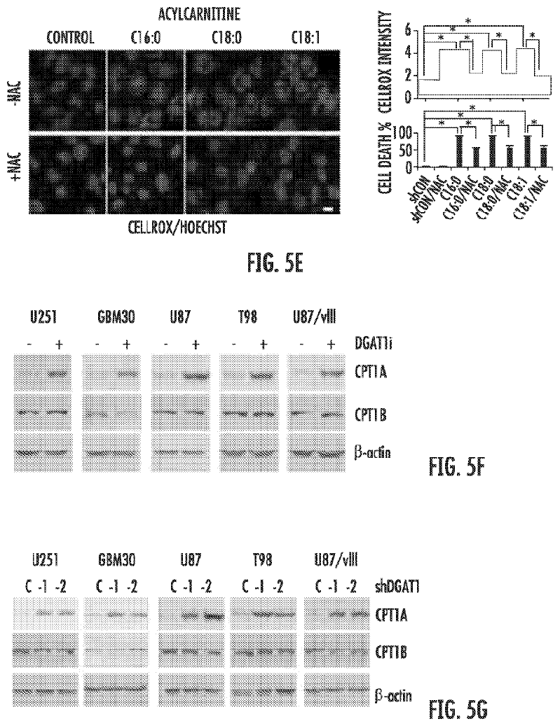

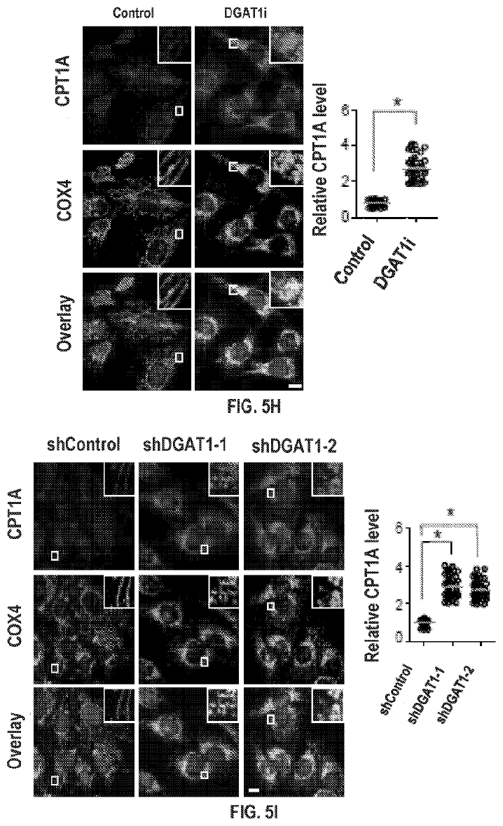

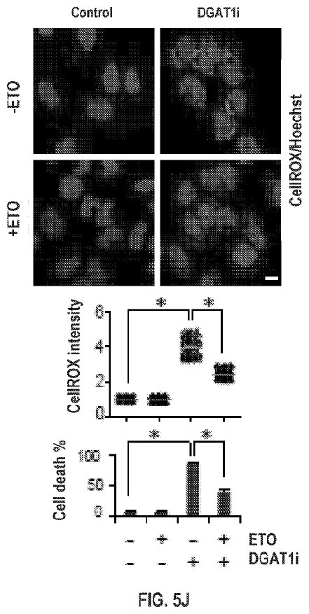

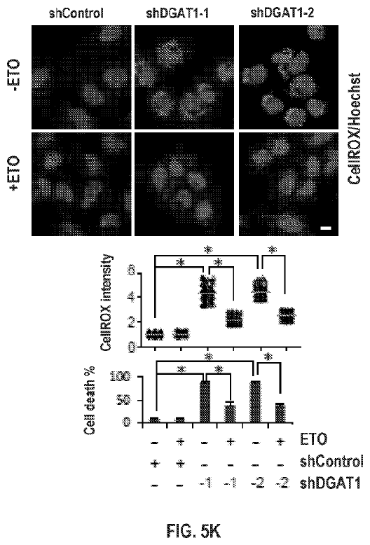

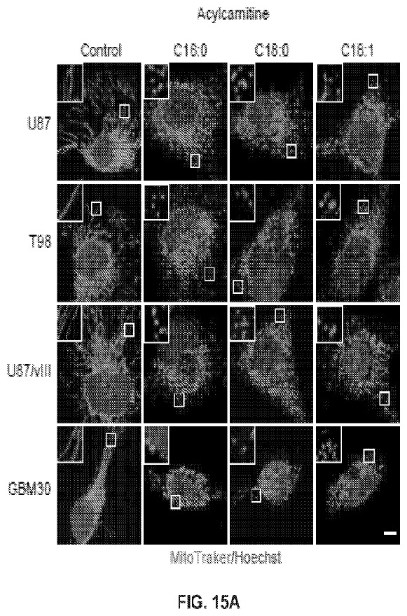

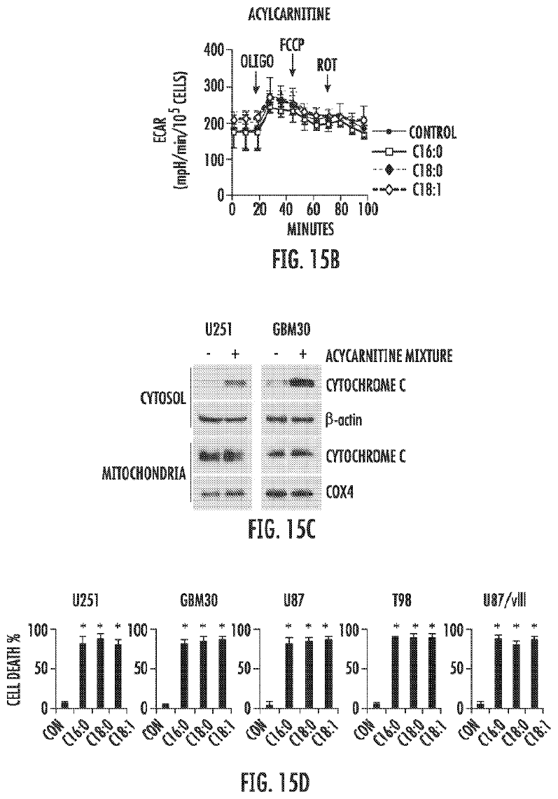

It was next examined whether increased acylcarnitines (FIG. 5a) cause toxicity to mitochondria and induce GBM cell death. GBM cells were supplemented with C16:0-, C18:0- or C18:1-carnitines for 24 hr. Similar with DGAT1 inhibition (FIG. 3), fluorescence imaging showed that all supplemented acylcarnitines strongly induced mitochondria fragmentation and swelling (FIG. 5b and FIG. 15a) and reduced oxygen consumption (FIG. 5c), while extracellular acidification rate (ECAR) was not affected (FIG. 15b). Moreover, these acylcarnitines also dramatically increased cytochrome c release (FIG. 15c), cleavage of the caspase 3, 9 and PARP proteins (FIG. 5d), and GBM cell death (FIG. 15d). Moreover, ROS levels were remarkably elevated by addition of any acylcarnitine (FIG. 5e, upper panels), while they did not dramatically induce ER stress in GBM cells, as shown by the stable levels of BIP and CHOP proteins (FIG. 15e). Moreover, neutralizing ROS with NAC significantly reduced acylcarnitine-induced GBM cell death (FIG. 5e). These data demonstrate that DGAT1 inhibition increases acylcarnitine levels, which could cause severe oxidative stress and kill GBM cells.

It was next examined whether inhibiting DGAT1 could affect CPT1 protein levels to allow the entry of excess fatty acids into mitochondria for oxidation. Western blotting showed that genetic or pharmacological inhibition of DGAT1 strongly increased CPT1A protein levels, while the levels of its isoform, CPT1B, were unchanged (FIG. 5f,g). IF imaging confirmed CPT1A elevation upon DGAT1 inhibition (FIG. 5h,i upper panels), and demonstrated that the increase in CPT1A localized to the mitochondria, which was stained by the marker cytochrome c oxidase subunit 4 (COX4) (Zeviani 1987), as seen by the overlapping yellow fluorescence (FIG. 5h,i, lower panels). It was then examined whether suppressing CPT1 activity with its inhibitor Etomoxir (ETO) (Weis 1994) could reduce ROS and GBM cell death caused by DGAT1 inhibition. Indeed, ETO treatment significantly reduced ROS and GBM cell death (FIG. 5j,k).

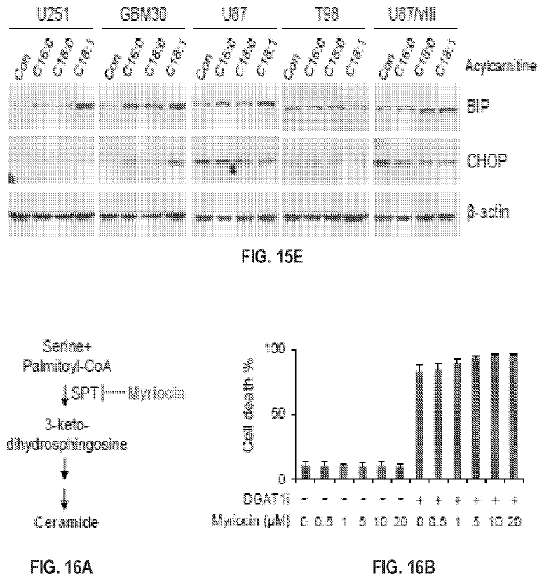

As DGAT1 inhibition induced a modest increase in ceramides (FIG. 4g), it was also tested whether they can contribute to GBM cell death by suppressing the activity of the upstream enzyme regulating ceramide synthesis, serine palmitoyltransferase (SPT), using its inhibitor myriocin (Holland 2007). The data showed that inhibition of ceramide synthesis had no rescue effects on DGAT1 inhibition-induced cell death (FIG. 16). These data suggest that the modest increase in ceramides in GBM cells does not play a major role in cell death induced by DGAT1 inhibition.

Genetic Inhibition of DGAT1 Significantly Suppresses Tumor Growth and Prolongs the Survival of GBM-Bearing Mice

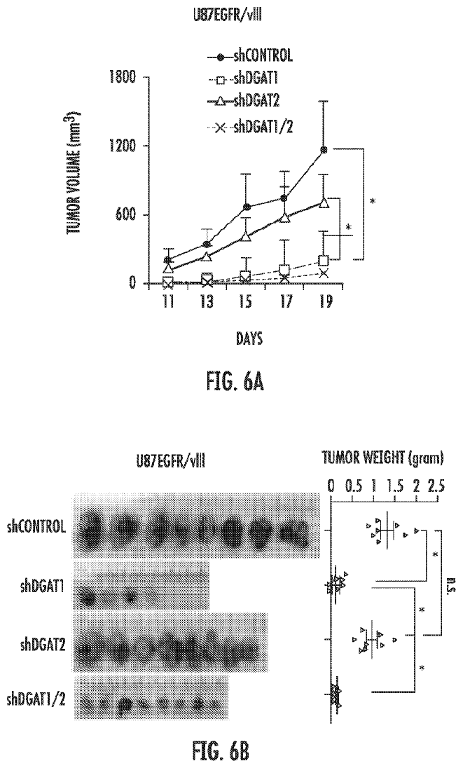

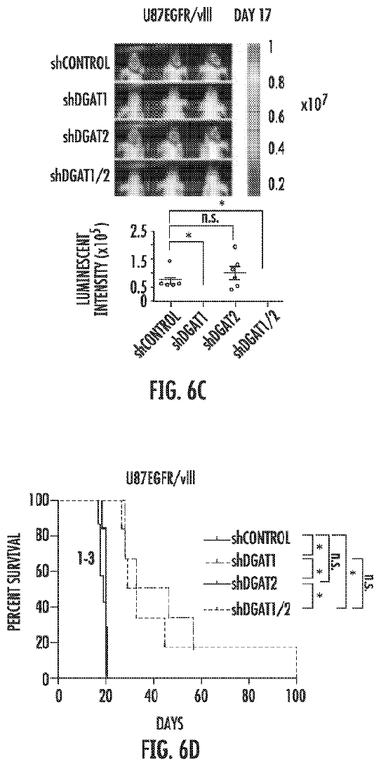

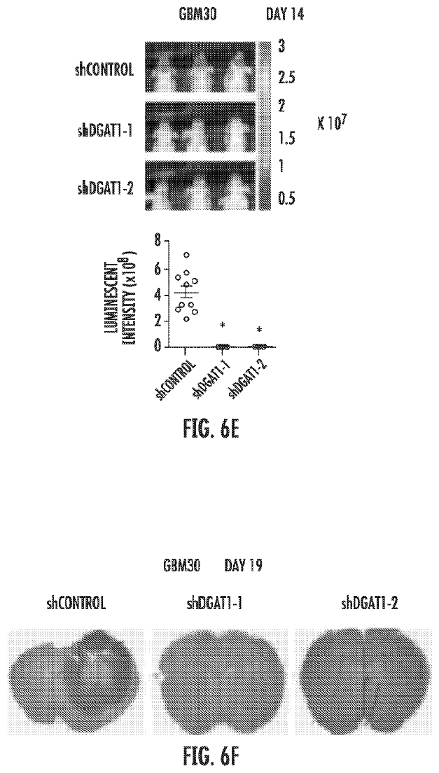

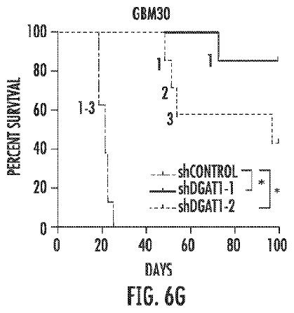

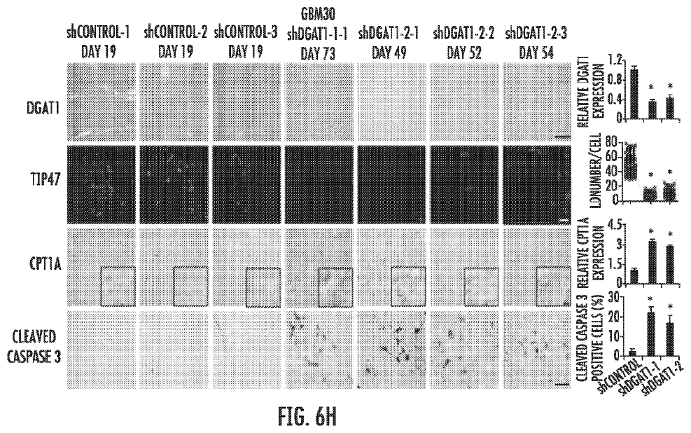

Next, the effects of genetic inhibition of DGAT1 and DGAT2 on tumor growth in GBM xenografts was examined. U87 cells were used that stably express luciferase (luc) and EGFRvIII, a constitutively active EGFR mutant, to generate a xenograft model (Gen 2016, Ru 2016; Cheng 2017). After shRNA knockdown of DGAT1 or DGAT2 for 48 hours, U87/EGFRvIII-luc cells were implanted into the mouse flank. Genetic inhibition of DGAT1 resulted in dramatic suppression of tumor growth, while only very minor inhibitory effects were observed with DGAT2 knockdown as compared to the control group (FIG. 6a,b). Moreover, no additional inhibitory effects on tumor growth were observed when both DGAT1 and DGAT2 were knocked down in comparison with DGAT1 inhibition alone (FIG. 6a,b).

These knockdown cells (48 hour shRNA infection) were transplanted in mouse brains and monitored tumor growth by bioluminescence imaging. The data showed that the control group developed large tumors in the mouse brain at day 17 after implantation, while no tumor growth was detected by bioluminescence imaging in the DGAT1 knockdown group (FIG. 6c). In contrast, knockdown of DGAT2 failed to inhibit tumor growth in mouse brains (FIG. 6c). Consequently, inhibition of DGAT1, but not DGAT2, significantly improved GBM-bearing mouse overall survival, and no additive effects were observed by knocking down both genes (FIG. 6d).

The effects of inhibition of DGAT1 in a primary GBM30-derived orthotopic xenograft model were then examined (Gen 2016; Ru 2016). After shRNA knockdown of DGAT1 for 48 hours, GBM30-luc cells were implanted into mice brains. Bioluminescence imaging taken at day 14 showed that DGAT1 inhibition markedly suppressed tumor growth as compared to the control group (FIG. 6e). The brains were excised at day 19 after implantation and sectioned to examine tumor size by H&E staining. Gross imaging showed that the tumor occupied almost half of the brain in the control group, while no tumor lesion was observed in the DGAT1 knockdown group at day 19 (FIG. 6f). As a consequence, knockdown of DGAT1 greatly prolonged the overall survival of GBM30-bearing mice (FIG. 6g).

DGAT1 expression, LDs formation, CPT1A protein level and apoptosis markers were examined in the tumor tissues from the brains of GBM30 xenografts, which were isolated from mice when they reached the mortality stage (FIG. 6g). IHC staining further showed that DGAT1 was significantly downregulated in the shRNA knockdown groups compared with control tumor (FIG. 6h, top panels), and LDs were markedly reduced, as assessed by IF staining of TIP47 (FIG. 6h). Moreover, as in the in vitro assay (FIG. 5f-i), CPT1A protein and cleaved caspase 3 were markedly elevated in tumor tissues from the DGAT1 knockdown groups (FIG. 6h). Together, these data demonstrate that genetic inhibition of DGAT1 to block fatty acid storage effectively suppressed tumor growth and induced apoptosis in GBM xenograft models.

Pharmacological Inhibition of DGAT1 Dramatically Suppresses GBM Tumor Growth and Induces Tumor Cell Apoptosis

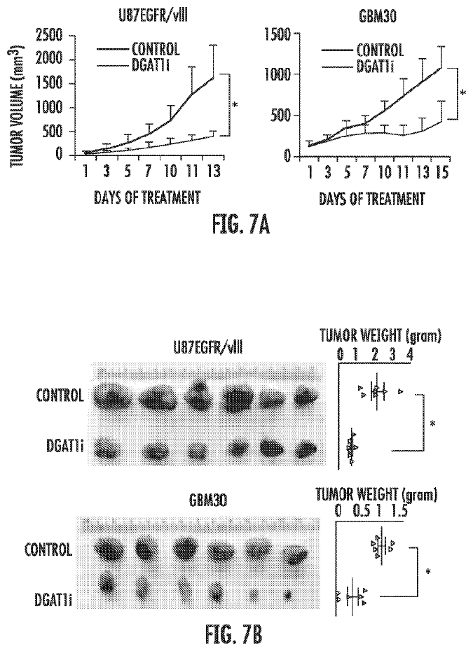

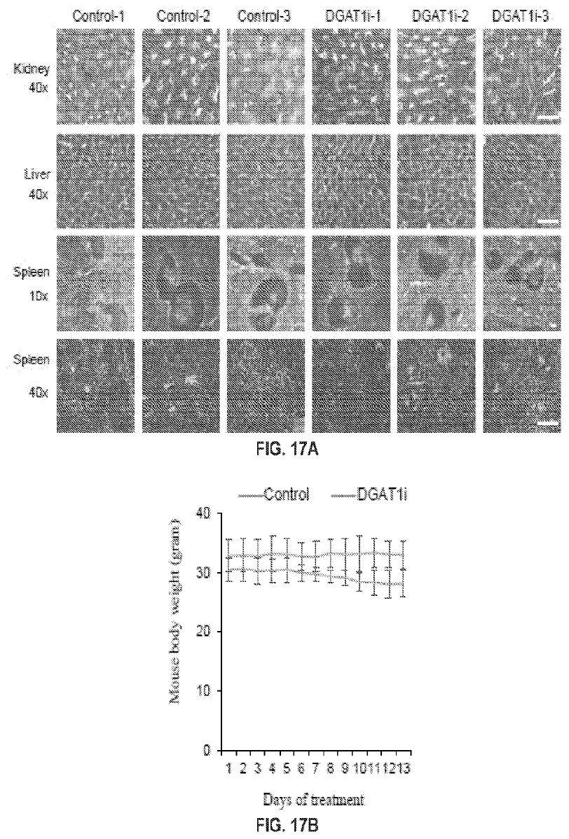

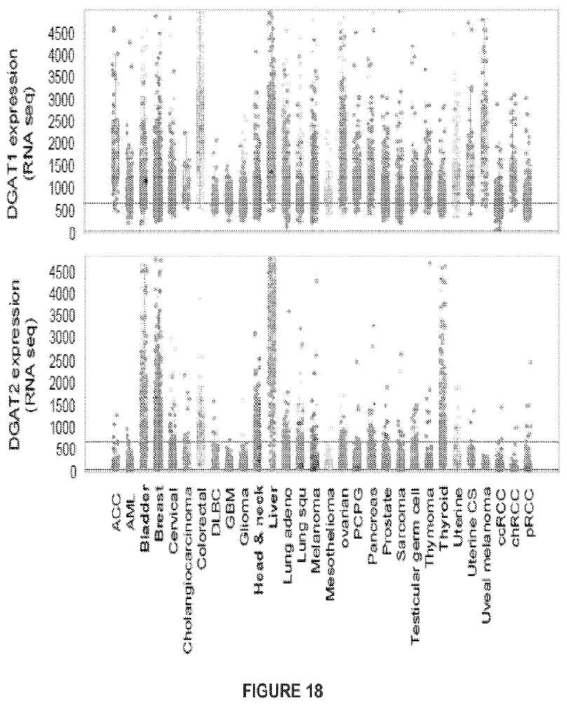

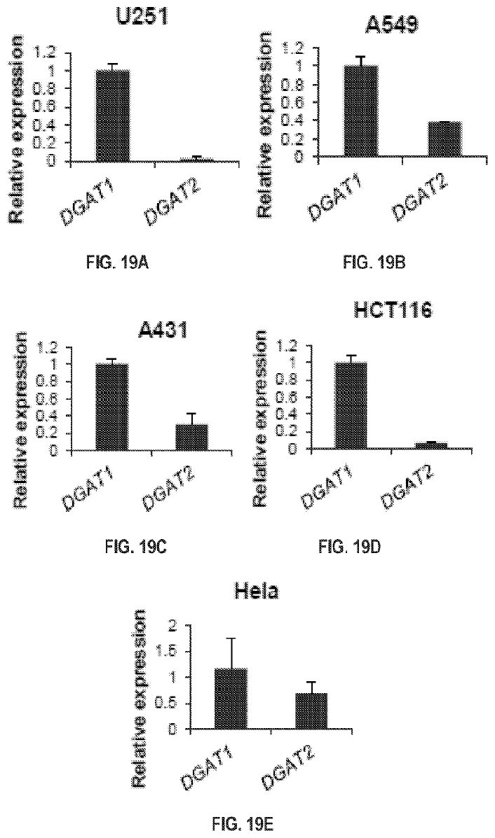

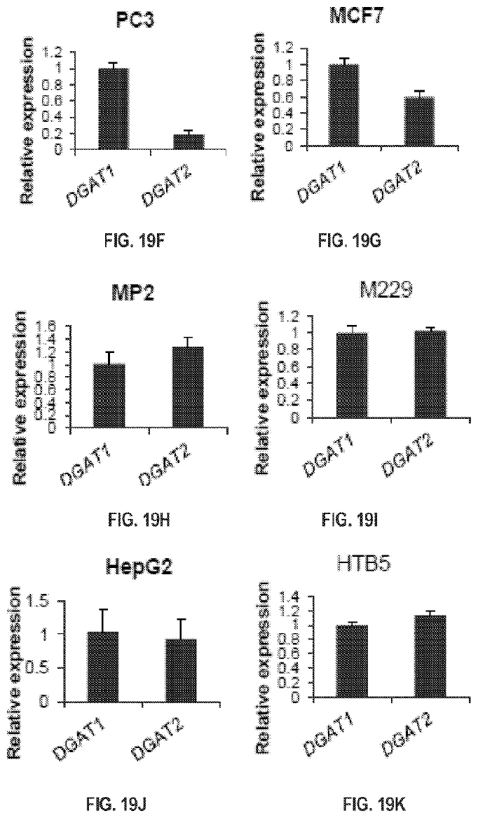

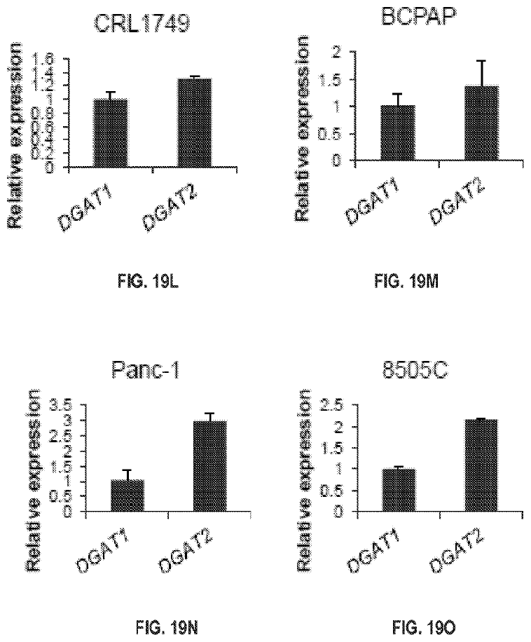

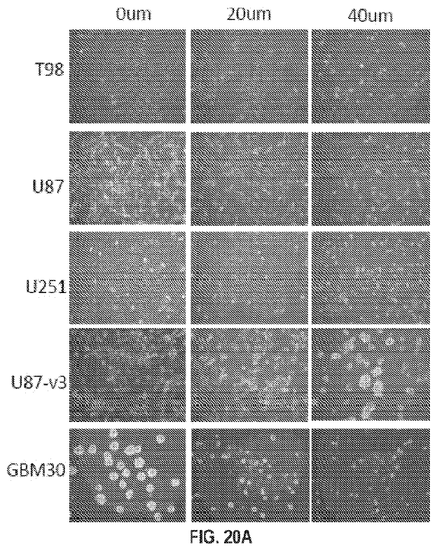

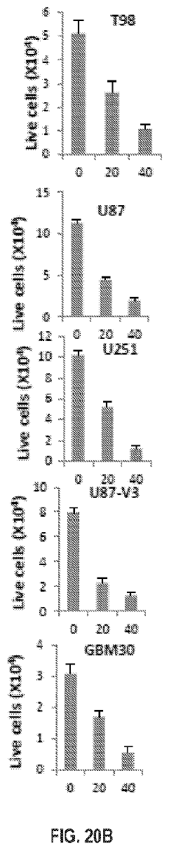

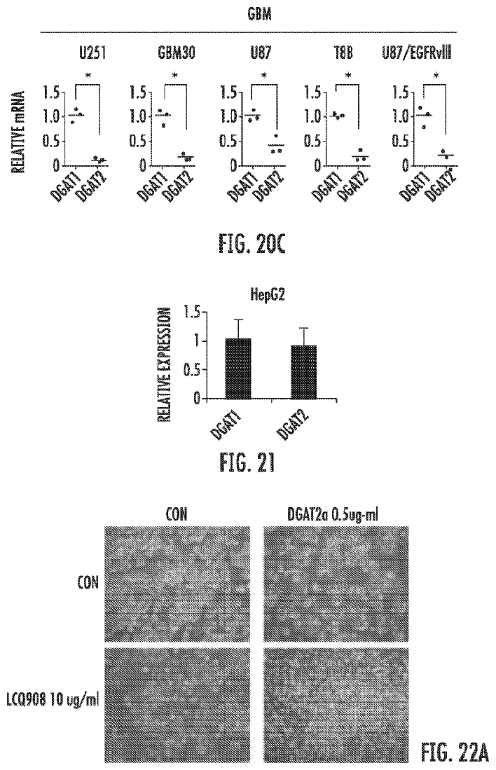

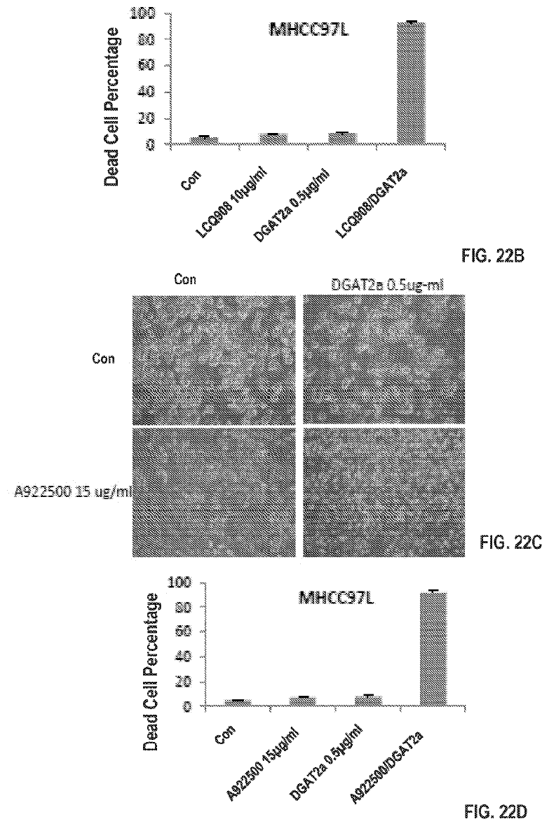

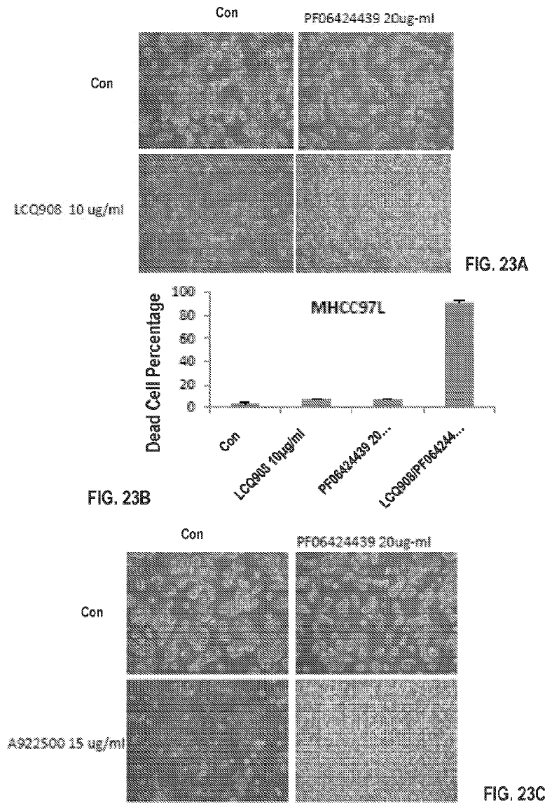

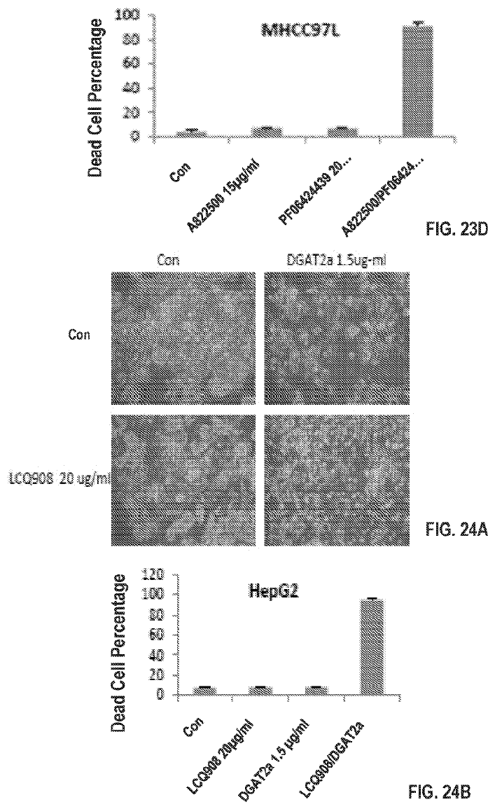

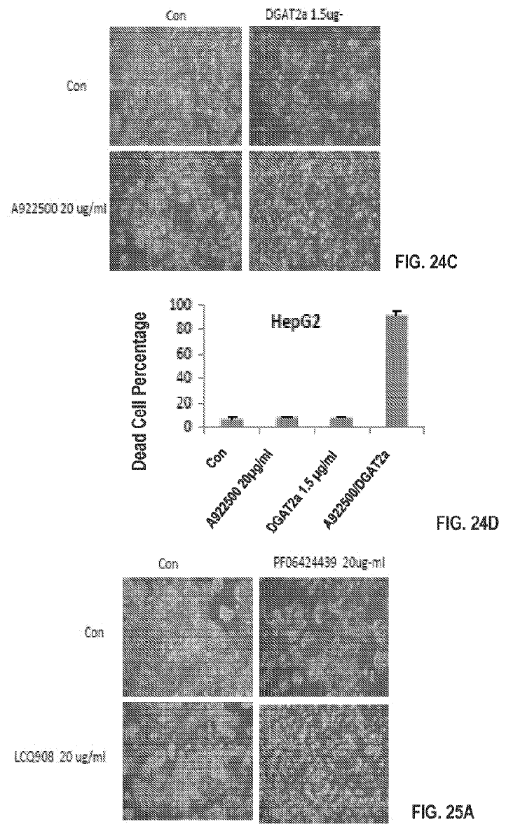

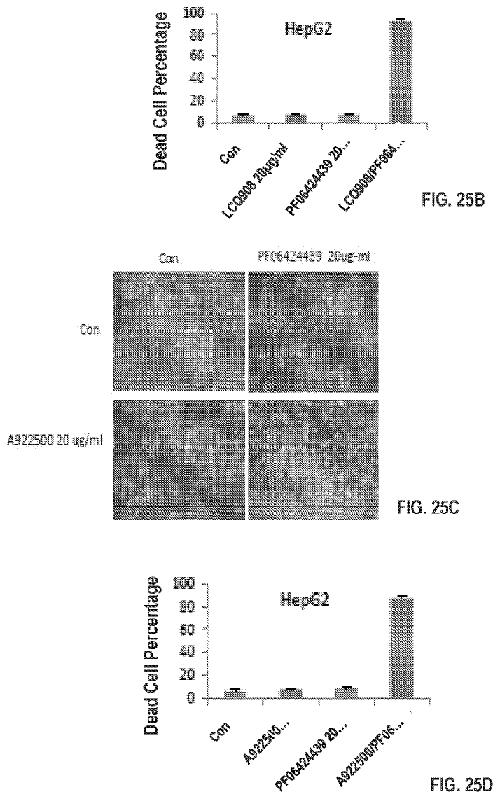

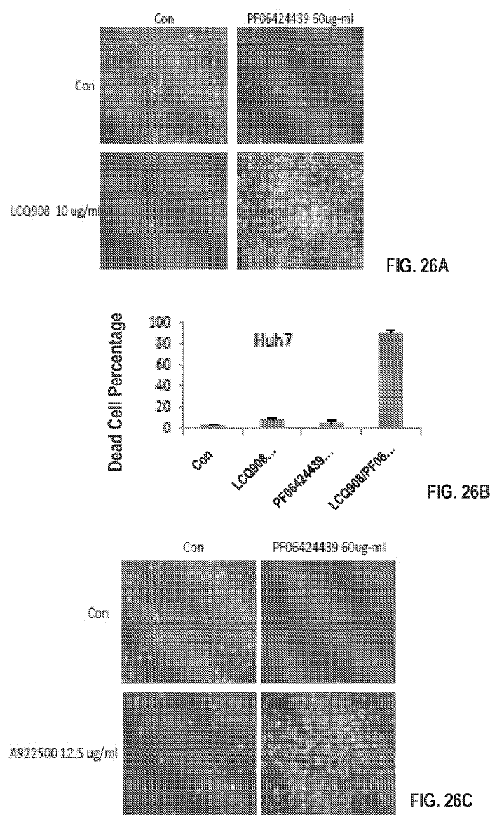

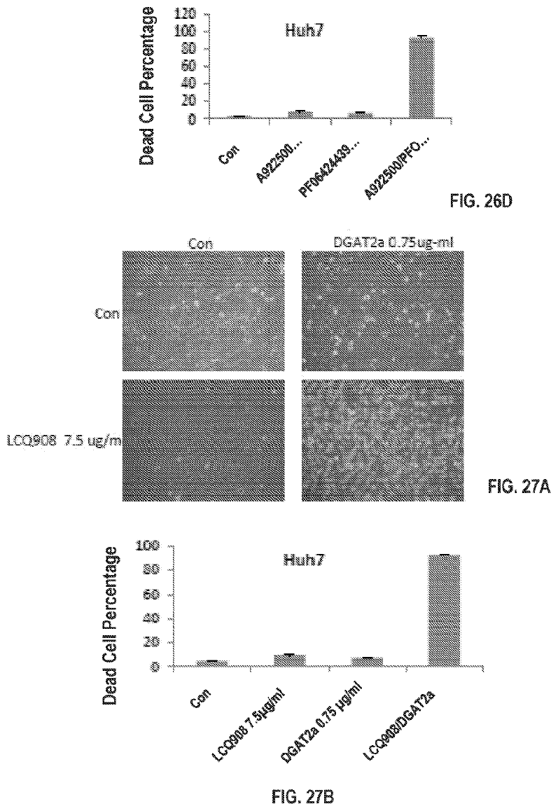

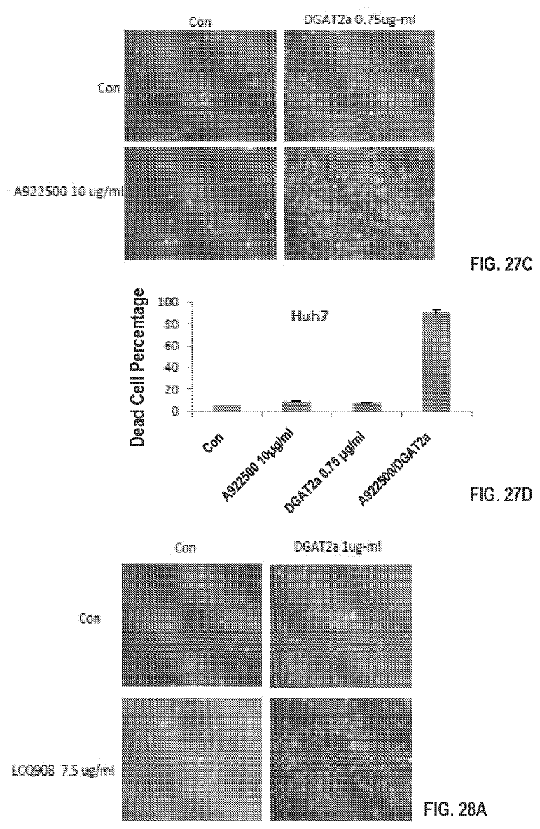

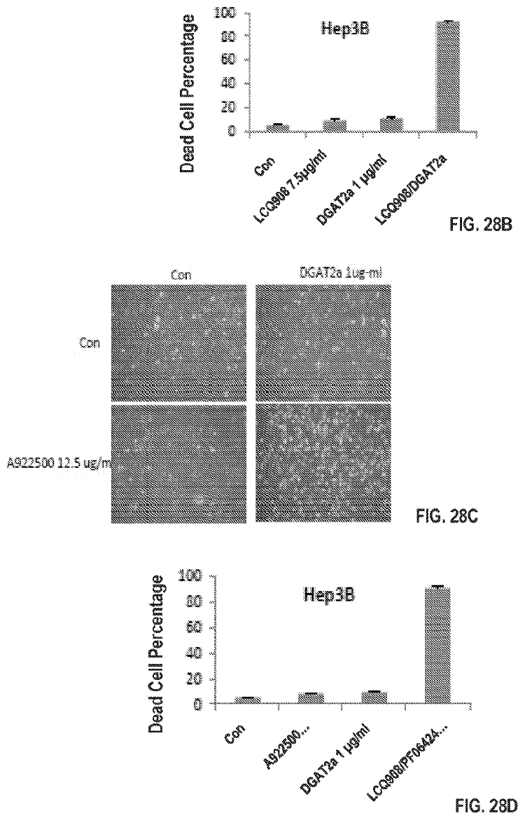

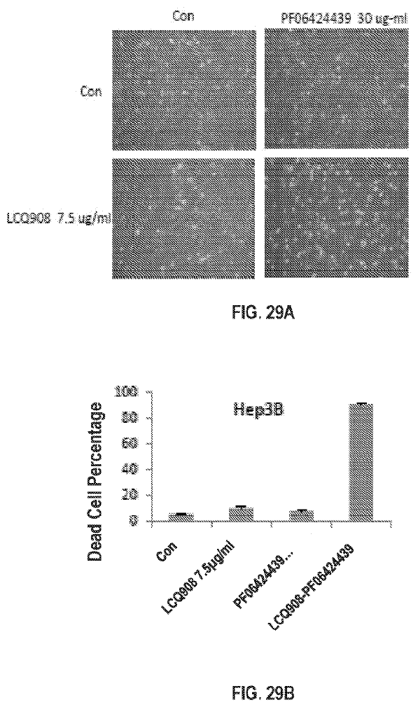

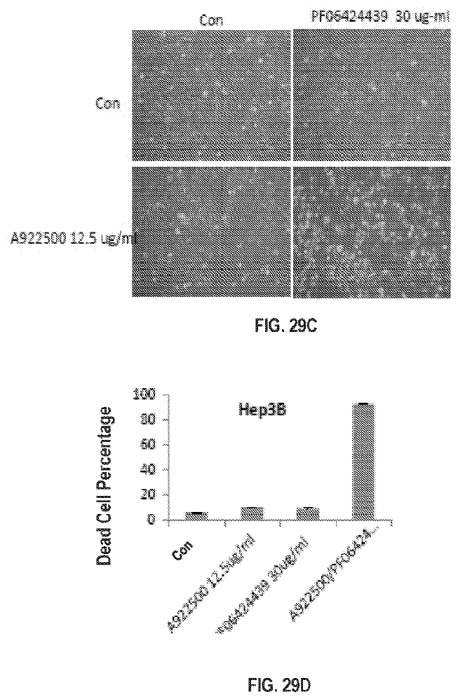

It was next examined whether pharmacological inhibition of DGAT1 is effective in inhibiting GBM growth using U87/EGFRvIII and GBM30 xenograft models. Tumor cells were implanted in mice flanks and started treatment with DGAT1 inhibitor A-922500 (120 mg/kg/day) when tumor reached approximately 80 mm3. The data showed that the DGAT1 inhibitor significantly suppressed tumor growth in both xenograft models (FIG. 7a), as further evidenced by the dramatic reduction in tumor weight (FIG. 7b). Moreover, as with genetic inhibition (FIG. 6h), pharmacological inhibition of DGAT1 dramatically suppressed LD formation, as shown by TIP47 (FIG. 7c), significantly increased CPT1A expression, and markedly elevated cleaved caspase 3 levels in tumor tissues (FIG. 7c). Importantly, we did not observe any toxic effects in mouse liver, kidney and spleen upon DGAT1 inhibitor treatment via H&E staining (FIG. 17a). The mice weight slightly decreased by about 8% after 13 days of treatment in comparison with the control group treated with vehicle (FIG. 17b), which could be caused by inhibition of TG formation in fat tissues. Together, these data demonstrate that pharmacological inhibition of DGAT1 is effective to suppress GBM tumor growth, while it does not induce noticeable toxic effects in mice.

Discussion

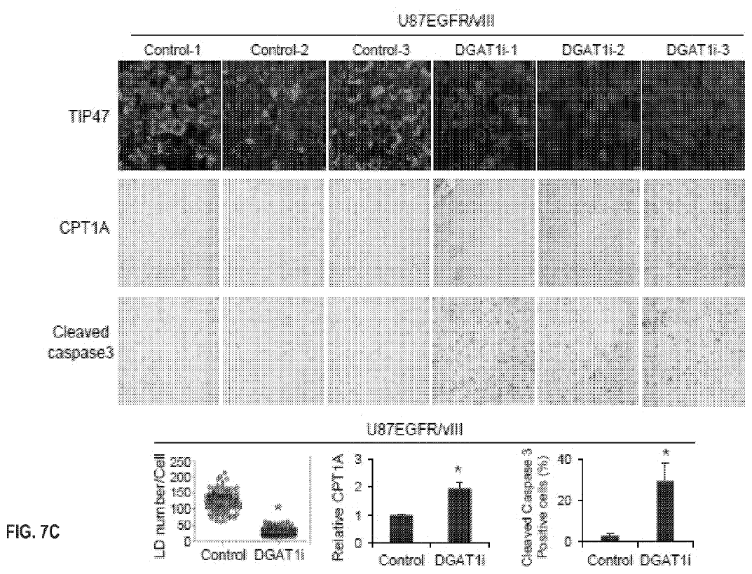

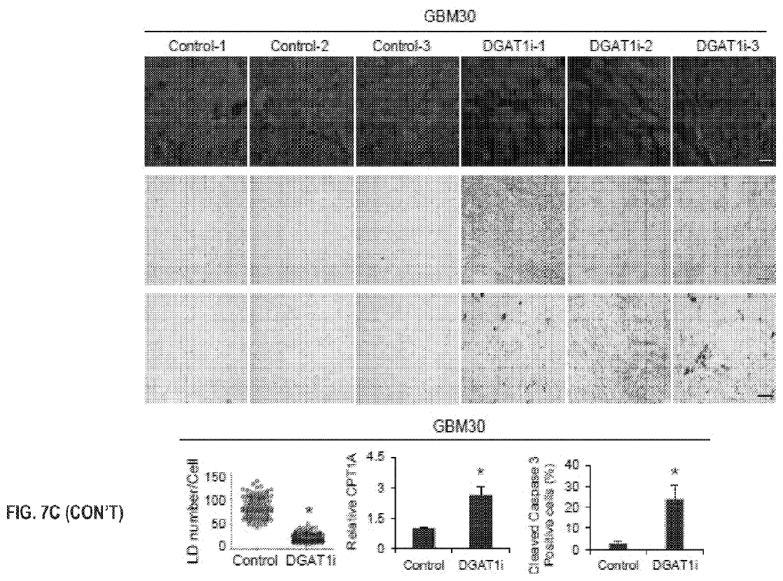

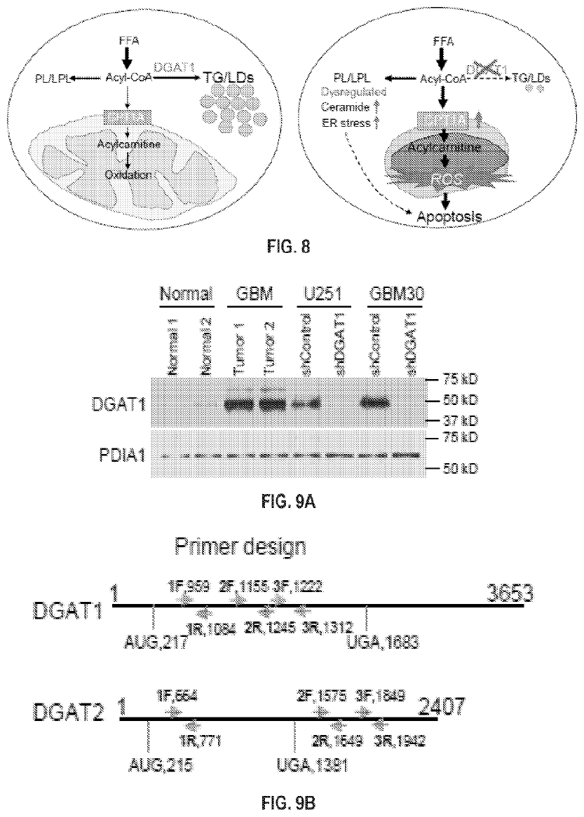

Lipid metabolism alterations are known to occur in various cancers (Cheng 2018, Accioly 2008, Cheng 2018 (2), Currie 2013) but the mechanisms by which tumor cells regulate lipid homoeostasis to prevent potential lipotoxicity have rarely been studied. In the present study, it was demonstrated that GBM activates the DGAT1-TG synthesis pathway to store excess fatty acids into LDs, thereby keeping the homeostasis of structural lipid synthesis and fatty acid oxidation (FIG. 8). Inhibiting DGAT1 significantly disrupts lipid homeostasis and results in excessive fatty acids shuttling into the mitochondria, leading to severe oxidative stress that kills GBM cells (FIG. 8). It was further shown that both genetic and pharmacological inhibition of DGAT1 effectively suppress tumor growth in GBM xenograft models, demonstrating that targeting DGAT1 is a new approach for GBM therapy.

Of note, the data further show that inhibiting DGAT1 significantly upregulates the CPT1A protein, which can facilitate the entry of excess fatty acids into the mitochondria for oxidation, leading to a remarkable rise in ROS production. In turn, increased ROS can damage the mitochondria and impair their oxidative capacity, resulting in the accumulation of large amounts of acylcarnitines and acetyl-CoA that further increase oxidative stress, thereby leading to cytochrome c release from the damaged mitochondria and triggering irreversible apoptotic cell death. In addition, the data also show that DGAT1 suppression significantly increases the levels of PC and PE, the major membrane structural phospholipids that are synthesized through the Kennedy pathway, as well as their metabolites, i.e., LPC and LPE, while it decreases the levels of other types of phospholipids, i.e., PI, PS, PG, LPI, LPS, and LPG, demonstrating that inhibiting DGAT1-TG synthesis results in fatty acid redistribution into structural phospholipids and disruption of lipid homeostasis. Furthermore, the data show that inhibition of DGAT1 triggers ER stress and increases ceramide levels, demonstrating that targeting DGAT1 could cause multiple toxic effects in tumors. Although the data showed that mitochondrial damage and cytochrome c release are sufficient triggers to induce apoptotic cell death, we cannot exclude the potential contribution of increased ER stress and ceramides to cell death induced by DGAT1 inhibition. It is also possible that dysregulated lipid homeostasis, enhanced ER stress and ceramides work jointly to synergize with mitochondrial damage to markedly kill GBM cells (FIG. 8).

Over the past two decades, the prognosis for GBM has remained very dismal, with an average survival of only 12-15 months, despite aggressive treatment (Wen 2016). One of the main reasons for this limited progress is a lack of full understanding of GBM biology. It was previously uncovered that de novo fatty acid synthesis is greatly increased in GBM to support its rapid growth. Increased glucose in cancer cells was found to activate sterol regulatory element-binding protein-1 (SREBP-1), a master transcriptional factor that controls de novo fatty acid synthesis (Cheng 2018), promoting the conversion of excess glucose into fatty acids (Cheng 2015; Guo 2016). It seems counterintuitive for cancer cells to keep synthesizing new fatty acids while storing large amounts of them into TG/LDs. Nevertheless, synthesizing and storing excess fatty acids under rich nutrient conditions is a greatly advantageous means developed by malignant tumors. In the tumor microenvironment, nutrient levels are always fluctuant (Muir 2018). When nutrient levels decrease, tumor cells could then quickly utilize lipid droplets to release free fatty acids for energy production and structural lipid synthesis, allowing them to maintain tumor cell survival under harsh conditions. This mechanism has the advantage to quickly boost malignant tumor growth.

As fatty acid synthesis is active in the liver, therapeutically targeting this process for cancer patients is limited. Thus, identifying the metabolic processes uniquely operating in GBM and other malignancies, while inactive in normal brain and other organs, is necessary to develop specific antitumor therapy. In fact, these findings showing that lipid homeostasis is sustained in GBM by storing excess fatty acids into TG/LDs open up a new opportunity to treat this deadly cancer. Based on these results, targeting DGAT1 to block fatty acid storage can induce severe oxidative stress and disrupt lipid homeostasis in tumor cells, while sparing normal brain tissues where DGAT1 expression is very low. Moreover, both DGAT1 and DGAT2 are expressed in human liver and adipose tissues, two major sites synthesizing TG in humans (Cases 1998; Cases 2001; Harris 2011). Thus, when inhibiting DGAT1, DGAT2 could still maintain TG synthesis, thereby offsetting potential toxic effects. This concept is supported by a recent study using a DGAT1 inhibitor to treat mouse and cultured adipocytes, which showed that inhibiting DGAT1 alone did not cause any noticeable toxic effects in mouse and adipocytes under physiological conditions, and that only ER stress was induced in adipocytes when lipolysis was strongly stimulated (Chitraju 2017). Moreover, another recent study tested a DGAT1 inhibitor in cultured mouse embryo fibroblast (MEF) cells (Nguyen 2017). The data showed that inhibition of DGAT1 did not cause any toxic effect in MEF cells when cultured in normal medium, and only a minor reduction of the mitochondria membrane potential was observed under severe nutrient-deprivation condition (serum/amino acid-free and low glucose). Furthermore, no ROS or ER stress was induced, demonstrating that inhibiting DGAT1 does not have toxicity in normal cultured MEF cells (Nguyen 2017).

Interestingly, LDs are also formed in multiple other human cancers. The data show that DGAT1 and DGAT2 expression is similar in the liver cancer cell line HepG2, and inhibiting TG/LDs formation to trigger lipotoxic effects in these cells required concurrent inhibition of both enzymes. A more recent study reported that inhibiting both DGAT1 and DGAT2 enzymes is required to suppress tumor growth in a renal cell carcinoma cell line A498-derived xenograft model (Ackerman 2018).

The data further showed that pharmacological inhibition of DGAT1 with A-922500 effectively suppresses GBM tumor growth and induces tumor cell apoptosis in xenograft models, while no obvious toxicity was observed in treated mice. The data strongly demonstrate that DGAT1 is a very druggable target and developing new inhibitors that can readily cross the BBB may shift the current paradigm for GBM therapy. Importantly, targeting DGAT1 has been tested in non-cancer patients in the clinic (Naik 2014). Herein, this provides a quick path to translate DGAT1 inhibition to clinical testing in patients with GBM and other cancers expressing high levels of DGAT1 and LDs. Targeting DGAT1-TG synthesis could have high impact in various types of cancers and cancer therapy.

Methods

Reagents and Chemicals

Antibodies for Cleaved Caspase 3 (Cat #9661), Cleaved Caspase 9(Cat #9509), PARP (Cat #9532), COX4 (Cat #4850T), BiP (Cat #3177), CHOP (Cat #2895), Anti-mouse IgG HRP-linked antibody (Cat #7076) and Anti-rabbit IgG HRP-linked antibody (Cat #7074) were purchased from Cell Signaling (Danvers, MA). Antibody for 3-actin (Cat #A1978), control shRNA (Cat #SHC002), paraformaldehyde (Cat #P6148), glutaraldehyde solution (Cat #G5882), puromycin dihydrochloride (Cat #P8833), human EGF (Cat #E9644), Heparin (Cat #H3393), and Triton X-100 (Cat #T8787), Poly-L-lysine hydrobromide (Cat #P5899), Laminin (Cat #L2020), Stearoyl-L-carnitine (C18:0, Cat #61229), DGAT2 inhibitor PF06424439 (Cat #PZ0233), Oligomycin A (Cat #73351), Carbonyl cyanide 4-(trifluoromethoxy) phenylhydrazone (FCCP) (Cat #C2920), Rotenone (Cat #R8875) and N-Acetyl-L-cysteine (Cat #A7250) were purchased from Sigma (St. Louis, MO). Alexa Fluor 488 goat anti-rabbit IgG (Cat #A-11034), Alexa Fluor 568 Goat Anti-Rabbit IgG (Cat #A-11036), Alexa Fluor 568 Goat Anti-Mouse IgG (Cat #A-11004), Neurobasal medium (Cat #21103-049), and B-27 Supplement (50×)/minus vitamin A (Cat #12587-010), TrypLE Express Enzyme (1×), no phenol red (Cat #12604-021), were purchased from Life Technologies (Grand Island, NY). Recombinant Human FGF basic 145 aa (Cat #4114-TC-01M) and Etomoxir (Cat #4539) were purchased from R&D. Antibody for CPT1A (Cat #Ab128568), CPT1B (Cat #ab104662), TIP47 (Perilipin 3; Cat #ab47638) were purchased from Abcam (Cambridge, MA). Antibodies for DGAT1 (Cat #sc-32861) and protein disulfide isomerase A1 (PDIA1, Cat #sc-30932) were purchased from Santa Cruz Biotechnology (Dallas, TX). DGAT1 inhibitor A922500 (Cat #959122-11-3) was from Cayman. Mito Tracker Red (Cat #M22425) and Green (Cat #M7514), the Cytochrome c antibody (Cat #BDB556433) were purchased from Thermo Fisher Scientific. CellROX Deep Red (Cat #C10422) was from Thermo Fisher Scientific. C16:0 carnintine (Cat #870851) and C18:1(A9-cis) Carnitine (Cat #870852) were obtained from Avanti Polar Lipids.

Western Blotting Cells were lysed by RIPA buffer (Cat #NC9484499; Fisher Scientific) containing protease inhibitor cocktail (Cat #11836170001; Roche) and phosphatase inhibitor (Cat #04906845001; Roche). The proteins were separated by using 12% SDS-PAGE, and transferred onto a Hybond ECL nitrocellulose membrane (Cat #RPN3032D; GE Healthcare).

After blocking for 1.5 h in 5% nonfat milk diluted by Tris-buffered saline containing 0.1% Tween 20, the membranes were incubated with various primary antibodies, followed by secondary antibodies conjugated to horseradish peroxidase. The immunoreactivity was revealed by use of an ECL kit (Cat #RPN2106; Amersham Biosciences Co).

Cell Proliferation

The cells with a total of 0.5 to 2×104 cells were seeded in 12-well plates. Cells were counted using a hemocytometer, and dead cells were assessed using trypan blue solution (Cat #15250061) (Life Technologies).

Immunohistochemistry (IHC)

The IHC was performed as previously described in Geng 2016. Briefly, tissue sections were cut from paraffin blocks of biopsies. Tissue slides were placed in oven at 60° C. for half an hour and then deparaffinized in xylenes 3×5 min followed by dipping in graded alcohols (100%, 95%, 80% and 70%) three times for 2 min each. Slides were washed with distilled water (dH2O) 3×5 min and immersed in 3% hydrogen peroxide for 10 min followed by washing with dH2O. Slides were transferred into pre-heated 0.01M Citrate buffer (pH 6.0) in a steamer for 30 min, and then washed with dH2O and PBS after cooling. Slides were blocked with 3% BSA/PBS at room temperature for 1 h and then incubated with primary antibody overnight at 4° C., followed by incubating with secondary antibody including Biotinylated Anti-rabbit IgG (Cat #BA-1100; Vector labs) and Biotinylated Anti-mouse IgG (Cat #BA-2000; Vector labs) at room temperature for 30 min. After incubation with avidin-biotin complex (Cat #PK-4000; Vector labs) followed by washing 3×5 min with PBS and staining with DAB solution (Cat #SK-4105; Vector labs), slides were washed with tap water, counterstained with hematoxylin (Cat #H-3401; Vector labs) and dipped briefly in graded alcohols (70%, 80%, 95% and 100%), in xylenes 2×5 min. Finally, slides were mounted and imaged.

Preparation of Cell Membrane Fractions

Cell membrane were isolated as described previously in Nohturfft 1998. Briefly, cells were washed once with PBS and harvest by scraping. Cells were resuspended with a buffer containing 10 mM HEPES-KOH (pH 7.6), 10 mM KCl, 1.5 mM MgCl2, and 1 mM sodium EDTA, 1 mM sodium EGTA, 250 mM sucrose and a mixture of protease inhibitors, 5 μg/ml pepstatin A (Cat #P5318), 10 μg/mL leupeptin (Cat #L2884), 0.5 mmol/L Phenylmethanesulfonyl fluoride (PMSF) (Cat #P7626), 1 mmol/L DTT (DL-Dithiothreitol) (Cat #43819), and 25 μg/mL ALLN (Calpain Inhibitor I) (Cat #A6185), for 30 min on ice. Extracts were passed through a 22G×1½ needle 30 times and centrifuged at 890×g at 4° C. for 5 min to remove nuclei. The supernatant was centrifuged at 20,000×g for 20 min at 4° C. For subsequent western blot analysis (for DGAT1 and PDIA1 protein), the pellet was dissolved in 0.1 ml of SDS lysis buffer (10 mM Tris-HCl pH 6.8, 100 mM NaCl, 1% (v/v) SDS, 1 mM sodium EDTA, and 1 mM sodium EGTA) and designated “membrane fraction”. The membrane fraction was incubated at 37° C. for 30 min, and protein concentration was determined. 1 μl bromophenol blue solution (100×) was added before the samples were subjected to SDS-PAGE.

Mitochondria and Cytosol Fractionation

The mitochondrial proteins were prepared using Qproteome Mitochondria Isolation Kit (Qiagen, Cat #37612) following manufacturer's instructions. Briefly, cells were harvested and washed with PBS, and resuspended with Lysis buffer and incubated at 4° C. for 10 minutes. The cells were centrifuged at 1,000×g at 4° C. for 10 minutes, and the supernatant was used as the cytosolic fraction. The pellet was resuspended in Disruption buffer and disrupted by using a 21G needle and a syringe. Following a centrifugation at 1,000×g at 4° C. for 10 minutes, the supernatant was transferred to a new tube. The supernatant was centrifuged at 6,000×g at 4° C. for 10 minutes. The pellet containing mitochondria was resuspended in Mitochondria storage buffer, and centrifuged at 6,000×g at 4° C. for 20 minutes. The pellet was resuspended in Mitochondria storage buffer and protein concentration was determined.

Quantitative Real-Time PCR

Total RNA was isolated with TRIzol (Cat #15596; Life Technologies) according to the manufacturer's protocol, and cDNA was synthesized with iScript™ cDNA Synthesis Kit (Cat #170-8891; Bio-Rad). Quantitative real-time PCR was performed with iQ™ SYBR® Green Supermix (Cat #170-8882; Bio-Rad) using the Applied Biosystems (ABI, it was merged into Life Technologies) 7900HT Real-Time PCR System. The expression was normalized to the 36B4 housekeeping gene and calculated with the comparative method (2-ΔΔCt). The primers are listed below in Table 1 (SEQ ID NOS: 1-14, sequentially):

TABLE 1

| | | | SEQ |
|---|---|---|---|
| | Primer | | ID |
| Gene | Name | Sequence | NO. |
| DGATI | DGAT1-1F | 5′-CCTACCGCGATCTCTACTACTT-3′ | 1 |
| DGATI | DGAT1-1R | 5′-GGGTGAAGAACAGCATCTCAA-3′ | 2 |
| DGATI | DGAT1-2F | 5′-CATGGACTACTCACGCATCAT-3′ | 3 |
| DGAT1 | DGAT1-2R | 5′-GTGGAAGAGCCAGTAGAAGAAG-3′ | 4 |
| DGAT1 | DGAT1-3F | 5′-CATGGACTACTCACGCATCAT-3′ | 5 |
| DGAT1 | DGAT1-3R | 5′-GTGGAAGAGCCAGTAGAAGAAG-3′ | 6 |
| DGAT2 | DGAT2-1F | 5′-GCTGACCACCAGGAACTATATC-3′ | 7 |
| DGAT2 | DGAT2-1R | 5′-GGGAACTTCTTGCTCACTTCT-3′ | 8 |
| DGAT2 | DGAT2-2F | 5′-CTGTTCTAGGTGGTGGCTAAAT-3′ | 9 |
| DGAT2 | DGAT2-2R | 5′-CACTTCAGGAAGGGAAGAAGAG-3′ | 10 |
| DGAT2 | DGAT2-3F | 5′-AACTGCAGGACCAGTTTCTC-3′ | 11 |
| DGAT2 | DGAT2-3R | 5′-GAGCATTCCAGATGCCTACTAC-3′ | 12 |
| 36B4 | 36B4-F | 5′-AATGGCAGCATCTACAACCC-3′ | 13 |
| 36B4 | 36B4-R | 5′-TCGTTTGTACCCGTTGATGA-3′ | 14 |

Lentiviral Transduction

Mission pLKO.1-puro lentivirus vector containing shRNA (shDGAT1-1, TRCN0000236207; shDGAT1-2, TRCN0000036151; shDGAT2-1, TRCN0000005193; shDGAT2-2, TRCN0000424717), the non-mammalian shRNA control (Cat #SHCO02) were purchased from Sigma. The shRNA vector and packing plasmids pCMV-R8.74psPAX2 and the envelope plasmid pMD2.G were transfected into 293FT cells using the polyethylenimine (Cat #23966; Polysciences). The supermatant was harvested at 48 h and 72 h and concentrated using the Lenti-X Concentrator (Cat #631232; Clontech). The virus titer were quantified by real time PCR by using qPCR Lentivirus Titration (Titer) Kit (Cat #LV900, ABM). The lentiviral transduction was performed according to Sigma's MISSION protocol with polybrene (8 μg/mL; Cat #H9268, Sigma,). The GBM cells (U251, U87, T98, U87/EGFRvIII and GBM30) and liver cancer cells HepG2 were firstly infected with positive control pLKO.1-puro-CMV-TagRFP™ lentivirus to determine the multiplicity of infection (MOI). The cells were then infected with the same amount of shControl, shDGAT1 or shDGAT2 lentivirus with pLKO.1-puro-CMV-TagRFP™ lentivirus.

Mitochondrial Membrane Potential

After washing for 3 times using PBS, U251 cells were replaced with FluoroBrite™ DMEM (Life Technologies) containing 5% FBS supplemented with Rhodamine 123 (0.05 g/ml, Thermo Fisher Scientific) for 30 min to determine mitochondrial membrane potential. After washing twice with PBS, cells were then incubated with Hoechst 33342 for 30 min before confocal imaging. More than 100 cells were analyzed and the fluorescence was quantified by the Image J software.

Haemotoxylin and Eosin (H&E) Staining

Paraffin tissue sections were deparaffinized in xylene and rehydrated in degraded ethanol (100%, 95% and 70% ethanol), respectively. After washing with dH2O, slides were stained with hematoxylin and eosin solution in sequence followed by washing with dH2O. Then slides were dehydrated in degraded ethanol and immersed in xylene followed by mounting in Permount.

GBM Patient Biopsies

GBM patient biopsies were obtained from the Department of Pathology at OSU Medical Center. One half of each biopsy was snap-frozen in liquid nitrogen and stored at −800C, and the second half was embedded in paraffin. The use of GBM patient tissues was approved by the OSU Institutional Review Board.

Glioma Tissue Microarray

The Glioma tissue microarray (TMA) was from the University of Kentucky (UK). Institutional Review Board approval was obtained at UK prior to study initiation. The staining intensity of DGAT1 was graded as 0, 1+, 2+ or 3+ in tumor tissues. H-score was assigned using the following formula: H-score=[1×(% cells 1+)+2×(% cells 2+)+3×(% cells 3+)]×100.

Cell Lines

Human GBM cell lines (U251, T98, U87, U87/EGFRvIII) and the human liver cancer cell line (HepG2) were cultured in Dulbecco's modified Eagle's medium (DMEM, Corning Incorporated) supplemented with 5% FBS (Gemini Bio-Products). GBM30 cells, a primary GBM patient-derived cell line that was previously molecularly characterized and escribed56, were cultured in neurobasal medium supplemented with B-27 serum-free supplements, heparin (2 mg/ml), EGF (50 ng/ml), and fibroblast growth factor (FGF; 50 ng/ml). U87/EGFRvIII-luc and GBM30-luc cells stably expressing luciferase were previously described (Cheng 2015; Geng 2016). All cell lines were cultured in a humidified atmosphere of 5% C02 at 37° C.

Lipid Droplet Staining and Quantification

For live cells, lipid droplets were stained with BODIPY 493/503 (0.5 PM) (Cat #D-3922; Life Technologies) for 30 min and visualized by confocal microscopy (Carl Zeiss LSM510 Meta, 63×/1.4 NA oil). More than 30 cells were analyzed and LD numbers were quantified with the Image J software (NIH) in a 3D stack, as previously described in Geng 2016. Lipid droplets were identified in patient and xenograft tumor tissues using immunofluorescence and an antibody against TIP47. After antigen retrieval, sections were incubated with the TIP47 antibody, followed by incubation with an appropriate secondary antibody, and slides were then mounted and imaged.

Thin Layer Chromatography (TLC)

TLC was performed as previously described (Guo 2009). Total lipid extract was obtained by suspending cells or tissues in 2 ml PBS containing the protease inhibitor 0.1 mM PMSF and adding 4 ml chloroform/methanol (2:1 vol/vol) with 0.01% butylated hydroxytoluene (Sigma). The solution was vortexed and centrifuged at 1500×g for 5 min. The organic phase was collected, and 2.5 ml chloroform was added to the residual aqueous phase for additional lipid extraction, and the solution was vortexed and centrifuged at 1500×g for 5 min. The organic phases were then pooled, and dried with nitrogen. TLC was performed by spotting the total lipid extract dissolved in chloroform onto a 5-10 cm EMD TLC Silica Gel TLC plates (#16834-2, EMD Chemicals), and developed with hexane/diethyl ether/acetic acid (80:20:2, vol/vol/vol). Lipids were visualized with iodine vapor prior to imaging (Guo 2009; Watson 2006).

GBM Xenograft Mouse Models

Intracranial xenograft models were generated using female athymic nude (NCr-nu/nu) mice (6-8 weeks of age obtained from OSU Target Validation Shared Resource). U87EGFRvIII (1×10⁶) cells infected with shDGAT1-, shDGAT2- or scramble shRNA-expressing lentivirus for 48 hr (suspended in 100 μl PBS) were implanted into mice flank to generate subcutaneous model. For intracranial xenograft model, U87EGFRvIII-luc (1×10⁵) or GBM30-luc (1×10⁵) cells infected with shDGAT1-, shDGAT2- or scramble shRNA-expressing lentivirus for 48 hours (suspended in 5 μl PBS) were implanted into mouse brains. Mice were observed until they became moribund, at which point they were sacrificed. For drug treatment, U87EGFRvIII (1×10⁶ cells suspended in 100 μl PBS) or GBM30 cells (4×10⁶ suspended in 100 μl PBS and then mixed with 100 μl matrigel) were implanted in mouse flanks. DGAT1 inhibitor A-922500 (120 mg/kg/day, oral gavage) was formulated by 1% Tween 80 in PBS and administrated to mice when tumor size reached approximately 80 mm³.

Lipidomics analysis Cultured cells were collected from plates using trypsin, then centrifuged at 500×g for 5 min. Cell pellets were homogenized on ice with 0.3 ml of 0.1×PBS in 1.5 ml RNase-free pellet pestle tube (Kimble Chase) using Kontes microtube pellet pestle rods (Kimble Chase). The protein concentration of cell homogenate was quantified following the instruction of Pierce™ BCA protein assay kit (Thermo Scientific). Bovine serum albumin was used as protein standard. A certain amount of individual homogenate (equivalent to 0.4-1.0 mg protein) was accurately transferred into a disposable glass culture test tube. The same lipid internal standard mixture for quantitation of lipids was added prior to lipid extraction. Lipid extraction was performed using a modified Bligh and Dyer procedure, as described previously in Wang 2014. Lipid extracts were resuspended into 200 μl of chloroform/methanol (1:1 vol/vol) per mg protein, and flushed with nitrogen, capped, and stored at −20° C. till lipid analysis. For shotgun lipidomics, lipid extracts were further diluted to a final concentration of ˜500 fmol/l, and the mass spectrometric analysis was performed on QqQ mass spectrometer (Thermo Scientific TSQ Altis) and high-resolution, accurate-mass mass spectrometer (Thermo Scientific Q Exactive) equipped with automated nanospray device (TriVersa NanoMate, Advion Bioscience Ltd.), as previously described in Han 2008. Identification and quantification of the lipids were performed using a lipid analyzation software (Yang 09 Wang 2016). Data were normalized per mg protein.

Reactive Oxygen Species (ROS) Detection

The cell-permeant CellROX Deep Red dye (Thermo Fisher Scientific) is non-fluorescent in its reduced state, produces bright near-infrared fluorescence upon oxidation by ROS, and has been used to detect oxidative stress in cells. After washing cells 3 times with PBS, cells were placed into FluoroBrite™ DMEM containing 5% FBS (Life Technologies) supplemented with 0.5 μM CellROX Deep Red for ROS detection or co-stained for MitoTracker Green (Thermo Fisher Scientific) for 30 min. After washing twice with PBS, cells were then incubated with Hoechst33342 for 30 min before confocal imaging. More than 100 cells were analyzed and fluorescence was quantified by the Image J software.

Seahorse Analysis

The Seahorse XFe 24 Extracellular Flux Bioanalyzer (Agilent) was used to measure oxygen consumption rate (OCR) and extracellular acidification rate (ECAR) according to the manufacturer's protocol. After treatment with the DGAT1 inhibitor A-922500 (20 μg/ml for 24 hours) or shRNA infection (48 hours), cells were placed into fresh DMEM medium containing 10 mM glucose, 2 mM L-glutamine, and 1 mM sodium pyruvate and incubated for 1 hr. Three metabolic inhibitors were sequentially loaded into each well, i.e., oligomycin (1 μM), followed by carbonyl cyanide 4-trifluoromethoxy-phenylhydrazone (FCCP) (2 μM), followed by rotenone (2 μM).

Transmission Electronic Microscopy (TEM)

Cells were fixed for 30 min in 2.5% glutaraldehyde in 0.1M phosphate buffer, pH 7.4 containing 0.1 M sucrose, and post-fixed in 1% osmium tetroxide/phosphate buffer for 30 min at room temperature. The cells were stained en bloc with 1% uranyl acetate for 30 min, followed by dehydration in graded ethanol series 50%, 30%, 85%, 95%, 100%, 100%.

The cells were finally embedded in Eponate 12 resin. Sections (70 nm) were produced on a Leica EM UC6 ultramicrotome and stained with 2% uranyl acetate and Reynold's lead citrate. TEM was performed on a FEI Tecnai G2 Spirit BioTWIN TEM at 80 kV. Images were captured using an AMT camera.

Mouse Bioluminescence Imaging

Mice implanted with GBM cells expressing luciferase were injected intraperitoneally with a Luciferin (#122796; Perkin Elmer) solution (15 mg/mL in PBS, dose of 150 mg/kg) by an intraperitoneal route. The bioluminescence images were acquired using the IVIS Lumina system and analyzed by the Living Image software.

Statistical Analysis

For cell proliferation, and quantification of LDs and TG, mitochondrial length and loss of cristae, quantification of ROS and TMA, and OCR, data were analyzed by unpaired Student's t-test or one-way ANOVA. Gene expression of DGAT1 and DGAT2 in TCGA GBM cohort was compared by paired Student's t-test. Kaplan-Meier plot was used to visualize patient and mice overall survival and the difference in survivals was tested by log-rank test. Tumor volume and weight were analyzed by one-way ANOVA. Multiplicity for each experiment was adjusted by Holm's procedure to control type I error rate at 0.05. Data analysis was performed in profession statistics software SAS 9.4 (SAS, Inc; Cary, NC) or Prism 7.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative materials and method steps disclosed herein are specifically described, other combinations of the materials and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

### REFERENCES

1. Carracedo, A., Cantley, L. C. & Pandolfi, P. P. Cancer metabolism: fatty acid oxidation in the limelight. Nat Rev Cancer _{13, 227-232} (2013).
2. Currie, E., Schulze, A., Zechner, R., Walther, T. C. & Farese, R. V., Jr. Cellular fatty acid metabolism and cancer. CellMetab 18, 153-161 (2013).
3. Qu, Q., Zeng, F., Liu, X., Wang, Q. J. & Deng, F. Fatty acid oxidation and carnitine palmitoyltransferase I: emerging therapeutic targets in cancer. Cell Death Dis 7, e2226 (2016).
4. Ertunc, M. E. & Hotamisligil, G. S. Lipid signaling and lipotoxicity in metaflammation: indications for metabolic disease pathogenesis and treatment. J Lipid Res 57, 2099-2114 (2016).
5. Listenberger, L. L. et al. Triglyceride accumulation protects against fatty acid-induced lipotoxicity. Proc Natl Acad Sci USA 100, 3077-3082 (2003).
6. Cheng, C., Geng, F., Cheng, X. & Guo, D. Lipid metabolism reprogramming and its potential targets in cancer. Cancer Commun (Lond) 38, 27 (2018).
7. Guo, D., Bell, E. H., Mischel, P. & Chakravarti, A. Targeting SREBP-1-driven lipid metabolism to treat cancer. Curr Pharm Des 20, 2619-2626 (2014).
8. Menendez, J. A. & Lupu, R. Fatty acid synthase and the lipogenic phenotype in cancer pathogenesis. Nat Rev Cancer 7, 763-777 (2007).
9. Geng, F. et al. Inhibition of SOAT1 Suppresses Glioblastoma Growth via Blocking SREBP-1-Mediated Lipogenesis. Clin Cancer Res 22, 5337-5348 (2016).
10. Geng, F. & Guo, D. Lipid droplets, potential biomarker and metabolic target in glioblastoma. Intern Med Rev (Wash D C) 3 (2017).
11. Wen, P. Y. & Reardon, D. A. Neuro-oncology in 2015: Progress in glioma diagnosis, classification and treatment. Nat Rev Neurol 12, 69-70 (2016).
12. Yue, S. et al. Cholesteryl ester accumulation induced by PTEN loss and PI3K/AKT activation underlies human prostate cancer aggressiveness. CellMetab 19, 393-406 (2014).
13. Qiu, B. et al. HIF2alpha-Dependent Lipid Storage Promotes Endoplasmic Reticulum Homeostasis in Clear-Cell Renal Cell Carcinoma. Cancer Discov 5, 652-667 (2015).
14. Accioly, M. T. et al. Lipid bodies are reservoirs of cyclooxygenase-2 and sites of prostaglandin-E2 synthesis in colon cancer cells. Cancer Res 68, 1732-1740 (2008).
15. Walther, T. C. & Farese, R. V., Jr. Lipid droplets and cellular lipid metabolism. Annu Rev Biochem 81, 687-714 (2012).
16. Krahmer, N., Farese, R. V., Jr. & Walther, T. C. Balancing the fat: lipid droplets and human disease. EMBO Mol Med 5, 973-983 (2013).
17. Coleman, R. A. & Lee, D. P. Enzymes of triacylglycerol synthesis and their regulation. Prog Lipid Res 43, 134-176 (2004).
18. Coleman, R. A. & Mashek, D. G. Mammalian triacylglycerol metabolism: synthesis, lipolysis, and signaling. Chem Rev 111, 6359-6386 (2011).
19. Yen, C. L., Stone, S. J., Koliwad, S., Harris, C. & Farese, R. V., Jr. Thematic review series: glycerolipids. DGAT enzymes and triacylglycerol biosynthesis. J Lipid Res 49, 2283-2301 (2008).
20. Cases, S. et al. Identification of a gene encoding an acyl CoA:diacylglycerol acyltransferase, a key enzyme in triacylglycerol synthesis. Proc Natl Acad Sci USA 95, 13018-13023 (1998).
21. Cases, S. et al. Cloning of DGAT2, a second mammalian diacylglycerol acyltransferase, and related family members. J Biol Chem 276, 38870-38876 (2001).
22. Cerami, E. et al. The cBio cancer genomics portal: an open platform for exploring multidimensional cancer genomics data. Cancer Discov 2, 401-404 (2012).
23. Gao, J. et al. Integrative Analysis of Complex Cancer Genomics and Clinical Profiles Using the cBioPortal. Science Signaling 6, pl1-pl1 (2013).
24. Ru, P. et al. Feedback Loop Regulation of SCAP/SREBP-1 by miR-29 Modulates EGFR Signaling-Driven Glioblastoma Growth. Cell Rep 16, 1527-1535 (2016).
25. Zhao, G. et al. Validation of diacyl glycerolacyltransferase I as a novel target for the treatment of obesity and dyslipidemia using a potent and selective small molecule inhibitor. Journal of medicinal chemistry 51, 380-383 (2008).
26. King, A. J. et al. In vivo efficacy of acyl CoA: diacylglycerol acyltransferase (DGAT) 1 inhibition in rodent models of postprandial hyperlipidemia. Eur J Pharmacol 637, 155-161 (2010).
27. Futatsugi, K. et al. Discovery and Optimization of Imidazopyridine-Based Inhibitors of Diacylglycerol Acyltransferase 2 (DGAT2). J Med Chem 58, 7173-7185 (2015).
28. Londhe, P. et al. Classical NF-κB Metabolically Reprograms Sarcoma Cells Through Regulation of Hexokinase 2. Frontiers in oncology 8, 104 (2018).
29. Cvrljevic, A. N. et al. Activation of Src induces mitochondrial localisation of de2-7EGFR (EGFRvIII) in glioma cells: implications for glucose metabolism. J Cell Sci 124, 2938-2950 (2011).
30. Liu, L. et al. Deregulated MYC expression induces dependence upon AMPK-related kinase 5. Nature 483, 608-612 (2012).
31. Aruoma, O. I., Halliwell, B., Hoey, B. M. & Butler, J. The antioxidant action of N-acetylcysteine: its reaction with hydrogen peroxide, hydroxyl radical, superoxide, and hypochlorous acid. Free Radic Biol Med 6, 593-597 (1989).
32. Lee, A. S. The ER chaperone and signaling regulator GRP78/BiP as a monitor of endoplasmic reticulum stress. Methods (San Diego, Calif) 35, 373-381 (2005).
33. Nishitoh, H. CHOP is a multifunctional transcription factor in the ER stress response. Journal of biochemistry 151, 217-219 (2012).
34. Cross, B. C. et al. The molecular basis for selective inhibition of unconventional mRNA splicing by an IRE1-binding small molecule. Proc Natl Acad Sci USA 109, E869-878 (2012).
35. Atkins, C. et al. Characterization of a novel PERK kinase inhibitor with antitumor and antiangiogenic activity. Cancer Res 73, 1993-2002 (2013).
36. Schreurs, M., Kuipers, F. & van der Leij, F. R. Regulatory enzymes of mitochondrial beta-oxidation as targets for treatment of the metabolic syndrome. Obesity reviews: an official journal of the International Association for the Study of Obesity 11, 380-388 (2010).
37. Zeviani, M. et al. Isolation of a cDNA clone encoding subunit IV of human cytochrome c oxidase. Gene 55, 205-217 (1987).
38. Weis, B. C., Cowan, A. T., Brown, N., Foster, D. W. & McGarry, J. D. Use of a selective inhibitor of liver carnitine palmitoyltransferase I (CPT I) allows quantification of its contribution to total CPT I activity in rat heart. Evidence that the dominant cardiac CPT I isoform is identical to the skeletal muscle enzyme. J Biol Chem 269, 26443-26448 (1994).
39. Holland, W. L. et al. Inhibition of ceramide synthesis ameliorates glucocorticoid-saturated-fat-, and obesity-induced insulin resistance. Cell Metab 5, 167-179 (2007).
33
40. Cheng, C. et al. Glucose-Mediated N-glycosylation of SCAP Is Essential for SREBP-1 Activation and Tumor Growth. Cancer Cell 28, 569-581 (2015).
41. Cheng, X., Li, J. & Guo, D. SCAP/SREBPs are Central Players in Lipid Metabolism and Novel Metabolic Targets in Cancer Therapy. Curr Top Med Chem 18, 484-493 (2018).
42. Currie, E., Schulze, A., Zechner, R., Walther, T. C. & Farese, R. V., Jr. Cellular Fatty Acid Metabolism and Cancer. Cell metabolism (2013).
43. Guo, D. SCAP links glucose to lipid metabolism in cancer cells. Mol Cell Oncol 3 (2016).
44. Guo, D., Bell, E. H. & Chakravarti, A. Lipid metabolism emerges as a promising target for malignant glioma therapy. CNS Oncol 2, 289-299 (2013).
45. Guo, D. et al. The AMPK agonist AICAR inhibits the growth of EGFRvIII-expressing glioblastomas by inhibiting lipogenesis. Proc Natl Acad Sci USA 106, 12932-12937 (2009).
46. Guo, D. et al. EGFR signaling through an Akt-SREBP-1-dependent, rapamycin-resistant pathway sensitizes glioblastomas to antilipogenic therapy. Sci Signal 2, ra82 (2009).
47. Guo, D. et al. An LXR agonist promotes glioblastoma cell death through inhibition of an EGFR/AKT/SREBP-1/LDLR-dependent pathway. Cancer Discov 1, 442-456 (2011).
48. Ru, P. & Guo, D. microRNA-29 mediates a novel negative feedback loop to regulate SCAP/SREBP-1 and lipid metabolism. RNA Dis 4 (2017).
49. Muir, A. & Vander Heiden, M. G. The nutrient environment affects therapy. Science 360, 962-963 (2018).
50. Harris, C. A. et al. DGAT enzymes are required for triacylglycerol synthesis and lipid droplets in adipocytes. J Lipid Res 52, 657-667 (2011).
51. Chitraju, C. et al. Triglyceride Synthesis by DGAT1 Protects Adipocytes from Lipid-Induced ER Stress during Lipolysis. Cell Metab 26, 407-418 e403 (2017).
52. Nguyen, T. B. et al. DGAT1-Dependent Lipid Droplet Biogenesis Protects Mitochondrial Function during Starvation-Induced Autophagy. Dev Cell 42, 9-21 e25 (2017).
53. Ackerman, D. et al. Triglycerides Promote Lipid Homeostasis during Hypoxic Stress by Balancing Fatty Acid Saturation. Cel/Rep 24, 2596-2605 e2595 (2018).
54. Naik, R. et al. Therapeutic strategies for metabolic diseases: Small-molecule diacylglycerol acyltransferase (DGAT) inhibitors. Chem Med Chem 9, 2410-2424 (2014).
55. Nohturfft, A., Brown, M. S. & Goldstein, J. L. Topology of SREBP cleavage-activating protein, a polytopic membrane protein with a sterol-sensing domain. J Biol Chem 273, 17243-17250 (1998).
56. Uchida, H. et al. Effective treatment of an orthotopic xenograft model of human glioblastoma using an EGFR-retargeted oncolytic herpes simplex virus. Mol Ther 21, 561-569 (2013).
57. Watson, A. D. Thematic review series: systems biology approaches to metabolic and cardiovascular disorders. Lipidomics: a global approach to lipid analysis in biological systems. J Lipid Res 47, 2101-2111 (2006).
58. Wang, M. & Han, X. Multidimensional mass spectrometry-based shotgun lipidomics. Methods Mol Biol 1198, 203-220 (2014).
59. Han, X., Yang, K. & Gross, R. W. Microfluidics-based electrospray ionization enhances the intrasource separation of lipid classes and extends identification of individual molecular species through multi-dimensional mass spectrometry: development of an automated high-throughput platform for shotgun lipidomics. Rapid Commun Mass Spectrom 22, 2115-2124 (2008).
60. Yang, K., Cheng, H., Gross, R. W. & Han, X. Automated lipid identification and quantification by multidimensional mass spectrometry-based shotgun lipidomics. Anal Chem 81, 4356-4368 (2009).
61. Wang, M., Wang, C., Han, R. H. & Han, X. Novel advances in shotgun lipidomics for biology and medicine. Prog Lipid Res 61, 83-108 (2016).

1. A method of treating cancer in a subject in need thereof, the method comprising administering to the subject an inhibitor of DGAT1, and an inhibitor of DGAT2, or a combination of both.

2. The method of claim 1, wherein the subject has liver cancer.

3. The method of claim 2, wherein the subject has been diagnosed with liver cancer prior to treatment.

4. The method of claim 1, wherein the subject is given a combination of DGAT1 and DGAT2 inhibitor.

5. The method of claim 1, wherein the DGAT1 inhibitor is Pradigastat.

6. The method of claim 1, wherein the DGAT1 inhibitor is A922500, T863, AZD-7687, or AZD 3988.

7. The method of claim 1, wherein the DGAT2 inhibitor is PF06424439.

8. The method of claim 1, wherein the DGAT2 inhibitor is JNJ DGAT2-A.

9. The method of claim 4, wherein the DGAT1 and DGAT2 inhibitors are given simultaneously.

10. The method of claim 4, wherein the DGAT1 inhibitor and the DGAT2 inhibitor are given sequentially.

11. The method of claim 10, wherein the DGAT1 and DGAT2 inhibitors are given within 24 hours of each other.

12. The method of claim 10, wherein the DGAT1 and DGAT2 inhibitors are given within 48 hours of each other.

13. The method of claim 1, wherein, in addition to DGAT1 and/or DGAT2 inhibition, the subject is also treated with a further liver cancer treatment method.

14. The method of claim 13, wherein said further liver cancer treatment method is selected from the group comprising one or more of surgery, ablation, embolization, radiation, targeted therapy, chemotherapy, and immunotherapy.

15. The method of claim 1, wherein the subject is monitored during treatment and the treatment is adjusted accordingly.

16. A kit comprising a DGAT1 inhibitor and a DGAT2 inhibitor.

17. The kit of claim 16, wherein the kit further comprises a means for delivering the DGAT1 and DGAT2 inhibitors to a subject in need thereof.

18. The kit of claim 16, wherein the DGAT1 and DGAT2 inhibitors are premixed together.

19-25. (canceled)


SEQUENCE LISTING (14 sequences)

SEQ ID NO: 1 - DNA, 22 bp, Artificial Sequence
  Synthetic Construct
cctaccgcgatctctactactt

SEQ ID NO: 2 - DNA, 21 bp, Artificial Sequence
  Synthetic Construct
gggtgaagaacagcatctcaa

SEQ ID NO: 3 - DNA, 21 bp, Artificial Sequence
  Synthetic Construct
catggactactcacgcatcat

SEQ ID NO: 4 - DNA, 22 bp, Artificial Sequence
  Synthetic Construct
gtggaagagccagtagaagaag

SEQ ID NO: 5 - DNA, 21 bp, Artificial Sequence
  Synthetic Construct
catggactactcacgcatcat

SEQ ID NO: 6 - DNA, 22 bp, Artificial Sequence
  Synthetic Construct
gtggaagagccagtagaagaag

SEQ ID NO: 7 - DNA, 22 bp, Artificial Sequence
  Synthetic Construct
gctgaccaccaggaactatatc

SEQ ID NO: 8 - DNA, 21 bp, Artificial Sequence
  Synthetic Construct
gggaacttcttgctcacttct

SEQ ID NO: 9 - DNA, 22 bp, Artificial Sequence
  Synthetic Construct
ctgttctaggtggtggctaaat

SEQ ID NO: 10 - DNA, 22 bp, Artificial Sequence
  Synthetic Construct
cacttcaggaagggaagaagag

SEQ ID NO: 11 - DNA, 20 bp, Artificial Sequence
  Synthetic Construct
aactgcaggaccagtttctc

SEQ ID NO: 12 - DNA, 22 bp, Artificial Sequence
  Synthetic Construct
gagcattccagatgcctactac

SEQ ID NO: 13 - DNA, 20 bp, Artificial Sequence
  Synthetic Construct
aatggcagcatctacaaccc

SEQ ID NO: 14 - DNA, 20 bp, Artificial Sequence
  Synthetic Construct
tcgtttgtacccgttgatga